(19) United States
(12) Patent Application Publication
(10) Pub. No.: US 2013/0224137 A1
(43) Pub. Date: Aug. 29, 2013

(54) SKIN LIGHTENING COMPOSITIONS

(71) Applicant: Lasya, Inc., Cininnati, OH (US)

(72) Inventors: Kalla Lynn Kvalnes, Cambridge, MD (US); Kevin R. Ewell, North Hollywood, CA (US); Mitchell A. deLong, Chapel Hill, NC (US); Moe Witwit, Porter Ranch, CA (US); Gilles Chambournier, Ann Arbor, MI (US)

(73) Assignee: LASYA, INC., Cininnati, OH (US)

(21) Appl. No.: 13/734,681

(22) Filed: Jan. 4, 2013

Related U.S. Application Data
(60) Provisional application No. 61/583,247, filed on Jan. 5, 2012.

(51) Int. Cl. A61K 8/60; A61K 8/49; C07H 15/203; C07D 313/04; C07D 319/12; C07D 313/18; C07D 311/20; C07D 215/227; C07D 335/02; C07D 333/32; C07D 333/48; C07D 333/64; A61K 8/34; C07C 43/23; A61Q 19/02

(52) U.S. Cl. CPC A61K 8/602; A61Q 19/02; A61K 8/4973; A61K 8/498; A61K 8/4926; A61K 8/4986; C07H 15/203; C07D 313/04; C07D 319/12; C07D 313/18; C07D 311/20; C07D 215/227; C07D 335/02; C07D 333/32; C07D 333/48; C07D 333/64; A61K 8/347; C07C 43/23
USPC 424/62

(57) ABSTRACT

Compositions and methods for lightening mammalian skin are described herein.

### CROSS-REFERENCE TO RELATED APPLICATION

This application claims priority to U.S. Provisional Patent Application No. 61/583,247, filed on Jan. 5, 2012, the entire contents of which are hereby incorporated by reference.

### INTRODUCTION

Skin lightening is an important skin care need. This includes at least one of lightening of basal skin tone and hyperpigmented regions. However, many commercially available products and services have been designed to conceal imperfections, such opaque products that cover the skin and mask its visual appearance. Compositions that actually improve, rather than simply conceal, skin imperfections are desirable.

Tyrosinase is an enzyme that is present within the melanosomes in epidermal melanocytes and catalyzes the committed step in the formation of melanin from tyrosine. Tyrosinase has tyrosine hydroxylase activity, effecting the hydroxylation of tyrosine to 3,4-dihydroxyphenylalanine (DOPA), and also has oxidase activity, and oxidizes DOPA to DOPAquinone. DOPAquinone is then ultimately converted to the pigment melanin. Inhibition of tyrosinase may accordingly lead to skin lightening via inhibition of melanogenesis.

Binding of an inhibitor to the active site of tyrosinase may result in decreased melanin formation. Currently, there are several tyrosinase inhibitors in the marketplace, including hydroquinone, kojic acid and arbutin. However, there are disadvantages to each of these products, including marginal activity and/or bioavailability. Another inhibitor, hydroquinone, is easily oxidized in the presence of light and air, and may result in skin irritation.

There is a need for effective skin lightening agents that are at least one of efficacious, stable, bioavailable, oxidation-resistant, and which do not produce by-products that irritate the skin. Such agents may be easier to use, have improved shelf lives and require less frequent applications.

### SUMMARY

In a first aspect, the disclosure may provide a composition comprising:

a) a compound of formula I:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur; and

b) at least one of an antioxidant, a glycol and a solvent;

wherein the composition comprises less than 100 ppm O₂, and wherein the composition exhibits a change in L* value of less than about 10.0 when stored at ambient temperature and pressure for a period of time, wherein the period of time is at least about 2 days.

In some embodiments, Z is O. In some embodiments, A and B are taken together with the atoms to which they are attached to form an optionally substituted ring having from 4-9 member atoms. In some embodiments, A and B are taken to taken together with the atoms to which they are attached to form a ring having 6 member atoms.

In some embodiments, the compound has the formula I-a, I-b, I-c, I-d, I-e or I-f as defined and described herein. In some embodiments, the compound of formula I is a deoxyArbutin compound disclosed herein. In some embodiments, the compound of formula I is 4-(tetrahydro-2H-pyran-2-yloxy)phenol.

In some embodiments, the compound of formula I is present in the composition at a concentration of about 0.5 wt. % to about 10 wt. %. In some embodiments, the composition comprises an antioxidant and the antioxidant comprises at least one of ascorbic acid, tocopherol, butylated hydroxybenzoic acid, butylated hydroxytoluene, butylated hydroxyanisole, uric acid, gallic acid, sorbic acid, glutathione, and esters and salts of any thereof. In some embodiments, the composition comprises an antioxidant and the antioxidant comprises at least one of ascorbic acid or a salt thereof, ascorbyl phosphate or a salt thereof, ascorbyl palmitate or a salt thereof, tocopherol or a salt thereof, tocopheryl acetate and sodium metabisulfite. In some embodiments, the composition comprises at least two antioxidants. In some embodiments, the composition comprises at least three antioxidants. In some embodiments, the composition comprises at least four antioxidants. In some embodiments, the composition comprises at least five antioxidants. In some embodiments, the composition comprises an antioxidant and the antioxidant is present in an amount of about 0.01 wt. % to about 3.0 wt. %. In some embodiments, the composition comprises an antioxidant and the antioxidant is present in an amount of about 0.05 wt. % to about 0.6 wt. %. In some embodiments, the composition comprises a glycol and the glycol is present in an amount of from about 0.1 wt. % to about 10 wt. %.

In some embodiments, the composition comprises a glycol and the glycol comprises ethoxydiglycol. In some embodiments, the composition further comprises at least one additional component selected from emulsifiers, chelating agents, preservatives, solvents, conditioning agents, pH adjusters, anti-inflammatory agents, sunscreens, retinoids, anti-aging agents, exfoliants and anti-acne agents. In some embodiments, the composition comprises less than 15 ppm O₂. In some embodiments, the period of time is at least about 7 days. In some embodiments, the period of time is at least about 10 days. In some embodiments, the composition is substantially free of dyes and pigments. In some embodiments, the composition exhibits a change in L* value of less than about 5.0 over the period of time. In some embodiments, the composition is substantially free of hydroquinone.

In a second aspect, the disclosure may provide a composition comprising:

a) a compound of formula I:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur;

b) an antioxidant; and

c) ethoxydiglycol;

wherein the composition comprises less than 100 ppm O₂.

In some embodiments, Z is O. In some embodiments, A and B are taken together with the atoms to which they are attached to form an optionally substituted ring having from 4-9 member atoms. In some embodiments, A and B are taken to taken together with the atoms to which they are attached to form a ring having 6 member atoms.

In some embodiments, the compound has the formula I-a, I-b, I-c, I-d, I-e or I-f as defined and described herein. In some embodiments, the compound of formula I is a deoxyArbutin compound disclosed herein. In some embodiments, the compound of formula I is 4-(tetrahydro-2H-pyran-2-yloxy)phenol. In some embodiments, the compound of formula I is present in the composition at a concentration of about 0.5 wt. % to about 10 wt. %.

In some embodiments, the antioxidant comprises at least one of ascorbic acid, tocopherol, butylated hydroxybenzoic acid, butylated hydroxytoluene, butylated hydroxyanisole, uric acid, gallic acid, sorbic acid, glutathione, and esters and salts of any thereof. In some embodiments, the antioxidant comprises at least one of ascorbic acid or a salt thereof, ascorbyl phosphate or a salt thereof, ascorbyl palmitate or a salt thereof, tocopherol or a salt thereof, tocopheryl acetate and sodium metabisulfite. In some embodiments, the composition comprises at least two antioxidants. In some embodiments, the composition comprises at least three antioxidants. In some embodiments, the composition comprises at least four antioxidants. In some embodiments, the composition comprises at least five antioxidants. In some embodiments, the antioxidant is present in an amount of about 0.01 wt. % to about 3.0 wt. %. In some embodiments, the antioxidant is present in an amount of about 0.05 wt. % to about 0.6 wt. %.

In some embodiments, ethoxydiglycol is present in an amount of from about 0.1 wt. % to about 10 wt. %. In some embodiments, the composition further comprises at least one additional component selected from emulsifiers, chelating agents, preservatives, solvents, conditioning agents, pH adjusters, anti-inflammatory agents, sunscreens, retinoids, anti-aging agents, exfoliants and anti-acne agents. In some embodiments, the composition comprises less than 15 ppm O₂.

In some embodiments, the composition exhibits a change in L* value of less than about 10.0 when stored at ambient temperature and pressure for a period of time, wherein the period of time is at least about 2 days. In some embodiments, the period of time is at least about 7 days. In some embodiments, the period of time is at least about 10 days. In some embodiments, the composition is substantially free of dyes and pigments. In some embodiments, the composition exhibits a change in L* value of less than about 10.0 over a period of time, wherein the period of time is at least about 2 days.

In a third aspect, the disclosure may provide a composition comprising:

a) about 0.5 wt. % to about 10 wt. % of a compound of formula I:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur;

b) about 0.01 wt. % to about 3.0 wt. % of at least one antioxidant;

c) about 0.1 wt. % to about 10 wt. % of at least one glycol;

d) about 0.01 wt. % to about 10 wt. % of at least one emulsifier; and

e) water;

wherein the composition comprises less than 100 ppm O₂.

In some embodiments, the compound has the formula I-a, I-b, I-c, I-d, I-e or I-f as defined and described herein. In some embodiments, the compound of formula I is a deoxyArbutin compound disclosed herein. In some embodiments, the compound of formula I is 4-(tetrahydro-2H-pyran-2-yloxy)phenol.

In some embodiments, the composition comprises about 1.0 wt. % to about 5.0 wt. % of the compound of formula I. In some embodiments, the composition comprises about 0.05 wt. % to about 0.6 wt. % of at least one antioxidant. In some embodiments, the at least one antioxidant is selected from the group consisting of ascorbic acid or a salt thereof, ascorbyl phosphate or a salt thereof, ascorbyl palmitate or a salt thereof, tocopherol or a salt thereof, tocopheryl acetate, butylated hydroxybenzoic acid, butylated hydroxytoluene, butylated hydroxyanisole, and sodium metabisulfite. In some embodiments, the composition comprises at least two antioxidants selected from the group consisting of ascorbic acid or a salt thereof, ascorbyl phosphate or a salt thereof, ascorbyl palmitate or a salt thereof, tocopherol or a salt thereof, tocopheryl acetate, butylated hydroxybenzoic acid, butylated hydroxytoluene, butylated hydroxyanisole, and sodium metabisulfite. In some embodiments, the composition comprises at least three antioxidants selected from the group consisting of ascorbic acid or a salt thereof, ascorbyl phosphate or a salt thereof, ascorbyl palmitate or a salt thereof, tocopherol or a salt thereof, tocopheryl acetate, butylated hydroxybenzoic acid, butylated hydroxytoluene, and sodium metabisulfite.

In some embodiments, the composition comprises at least four antioxidants selected from the group consisting of ascorbic acid or a salt thereof, ascorbyl phosphate or a salt thereof, ascorbyl palmitate or a salt thereof, tocopherol or a salt thereof, tocopheryl acetate, butylated hydroxybenzoic acid, butylated hydroxytoluene, and sodium metabisulfite. In some embodiments, the composition comprises at least five antioxidants selected from the group consisting of ascorbic acid or a salt thereof, ascorbyl phosphate or a salt thereof, ascorbyl palmitate or a salt thereof, tocopherol or a salt thereof, tocopheryl acetate, butylated hydroxybenzoic acid, butylated hydroxytoluene, and sodium metabisulfite.

In some embodiments, the composition comprises about 0.5 wt. % to about 7.5 wt. % of at least one glycol. In some embodiments, the at least one glycol is selected from the group consisting of ethoxydiglycol and 1,3-butylene glycol. In some embodiments, the composition comprises ethoxydiglycol and 1,3-butylene glycol.

In some embodiments, at least one emulsifier is selected from the group consisting of fatty alcohols, fatty alcohol polyethylene glycol ethers, and acrylates/C₁₀-C₃₀ alkyl acrylate crosspolymers. In some embodiments, the at least one emulsifier is selected from the group consisting of cetyl alcohol, stearyl alcohol, steareth-2, steareth-21, and an acrylates/C₁₀-C₃₀ alkyl acrylate crosspolymer.

In some embodiments, the composition further comprises about 0.01 to about 5.0 wt. % of at least one chelator. In some embodiments, the at least one chelator is ethylenediaminetetraacetic acid.

In some embodiments, the composition further comprises about 0.01 to about 10 wt. % of at least one conditioning agent. In some embodiments, the at least one conditioning agent is selected from the group consisting of polypropylene glycol-14 butyl ether and dimethicone. In some embodiments, the composition comprises polypropylene glycol-14 butyl ether and dimethicone.

In some embodiments, the composition further comprises about 0.01 to about 5.0 wt. % of at least one preservative. In some embodiments, the at least one preservative is selected from the group consisting of benzyl alcohol, chlorphenesin, methyl paraben, butyl paraben, and polyaminopropyl biguanide. In some embodiments, the composition comprises at least two preservatives selected from the group consisting of benzyl alcohol, chlorphenesin, methyl paraben, butyl paraben, and polyaminopropyl biguanide. In some embodiments, the composition comprises at least three preservatives selected from the group consisting of benzyl alcohol, chlorphenesin, methyl paraben, butyl paraben, and polyaminopropyl biguanide. In some embodiments, the composition comprises benzyl alcohol, chlorphenesin, and polyaminopropyl biguanide.

In some embodiments, the composition further comprises at least one pH adjuster. In some embodiments, the pH adjuster is selected from the group consisting of triethanolamine, sodium hydroxide, hydrochloric acid and lactic acid. In some embodiments, the pH adjuster is selected from the group consisting of triethanolamine and lactic acid.

In some embodiments, the composition comprises less than 15 ppm O₂. In some embodiments, the composition exhibits a change in L* value of less than about 10.0 when stored at ambient temperature and pressure for a period of time, wherein the period of time is at least about 2 days. In some embodiments, the period of time is at least about 7 days. In some embodiments, the period of time is at least about 10 days. In some embodiments, the composition exhibits a change in L* value of less than about 5.0 over the period of time. In some embodiments, the composition is substantially free of dyes and pigments. In some embodiments, the composition is substantially free of hydroquinone.

In a fourth aspect, the disclosure may provide a method of lightening mammalian skin, the method comprising topically applying to mammalian skin a therapeutically effective amount of a composition as defined and described herein, such as a composition according to any of the first, second and third aspects of the disclosure. In some embodiments, the method comprises applying from about 0.1 g to about 10 g per cm² of the composition to the skin. In some embodiments, the composition is applied to the skin about once daily. In some embodiments, the composition is applied to the skin about twice daily.

In a fifth aspect, the disclosure may provide a method of preparing a composition, the method comprising:

mixing a compound of formula I:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur

with at least one of an antioxidant, a glycol and a solvent under an inert atmosphere; and

maintaining the pH at a range of about 6.0 to about 10.0.

In some embodiments, the temperature is from about 30° C. to about 80° C. during addition of the compound of formula I. In some embodiments, the temperature is from about 45° C. to about 60° C. during addition of the compound of formula I. In some embodiments, the solvent comprises water. In some embodiments, the solvent comprises an oil-based solvent. In some embodiments, the method further comprises adding at least one additional component selected from emulsifiers, chelating agents, preservatives, solvents, conditioning agents, anti-inflammatory agents, sunscreens, retinoids, anti-aging agents, exfoliants and anti-acne agents. In some embodiments, the inert atmosphere comprises nitrogen. In some embodiments, the inert atmosphere comprises argon. In some embodiments, Z is O. In some embodiments, A and B are taken together with the atoms to which they are attached to form an optionally substituted ring having from 4-9 member atoms. In some embodiments, A and B are taken to taken together with the atoms to which they are attached to form a ring having 6 member atoms.

In some embodiments, the compound has the formula I-a, I-b, I-c, I-d, I-e or I-f as defined and described herein. In some embodiments, the compound of formula I is a deoxyArbutin compound disclosed herein. In some embodiments, the compound of formula I is 4-(tetrahydro-2H-pyran-2-yloxy)phenol.

In some embodiments, the compound of formula I is added to the composition at a concentration of about 0.5 wt. % to about 10 wt. %. In some embodiments, the antioxidant comprises at least one of ascorbic acid, tocopherol, butylated hydroxybenzoic acid, butylated hydroxytoluene, butylated hydroxyanisole, uric acid, gallic acid, sorbic acid, glutathione, and esters and salts of any thereof. In some embodiments, the antioxidant comprises at least one of ascorbic acid or a salt thereof, ascorbyl phosphate or a salt thereof, ascorbyl palmitate or a salt thereof, tocopherol or a salt thereof, tocopheryl acetate and sodium metabisulfite. In some embodiments, the method comprises adding at least two antioxidants. In some embodiments, the method comprises adding at least three antioxidants. In some embodiments, the method comprises adding at least four antioxidants. In some embodiments, the method comprises adding at least five antioxidants. In some embodiments, the antioxidant is added in an amount of about 0.01 wt. % to about 3.0 wt. %. In some embodiments, the antioxidant is added in an amount of about 0.05 wt. % to about 0.6 wt. %.

In some embodiments, the glycol is added in an amount of from about 0.1 wt. % to about 10 wt. %. In some embodiments, the method further comprises adding at least one additional component selected from emulsifiers, chelating agents, preservatives, solvents, conditioning agents, pH adjusters, anti-inflammatory agents, sunscreens, retinoids, anti-aging agents, exfoliants and anti-acne agents.

In some embodiments, composition comprises less than 100 ppm O₂. In some embodiments, the composition comprises less than 15 ppm O₂. In some embodiments, the composition exhibits a change in L* value of less than about 10.0 when stored at ambient temperature and pressure for a period of time, wherein the period of time is at least about 2 days. In some embodiments, the period of time is at least about 7 days. In some embodiments, the period of time is at least about 10 days. In some embodiments, the composition is substantially free of dyes and pigments. In some embodiments, the composition is substantially free of hydroquinone.

In a sixth aspect, the disclosure may provide a method of lightening mammalian hair, comprising topically applying to mammalian hair a therapeutically effective amount of a composition as defined and described herein, such as a composition according to any of the first, second and third aspects of the disclosure. In some embodiments, the method comprises applying from about 0.1 g to about 10 g per cm² of the composition to the hair. In some embodiments, the composition is applied to the hair about once daily. In some embodiments, the composition is applied to the hair about twice daily.

Other aspects and embodiments of the disclosure will be come apparent in light of the following description.

### BRIEF DESCRIPTION OF THE DRAWINGS

FIG. 1 is a graph illustrating the lightening of darker pigmented guinea pig skin upon treatment with a deoxyArbutin composition described herein.

FIG. 2 is a graph illustrating the lightening of medium pigmented guinea pig skin upon treatment with a deoxyArbutin composition described herein.

FIG. 3 illustrates the lightening of human skin upon treatment with a deoxyArbutin composition described herein.

FIG. 4 is a graph illustrating the change in skin lightness of a darker pigmented guinea pig following treatment with a deoxyArbutin composition described herein, after a period of twice-daily treatments, and after treatments were stopped.

FIG. 5 is a graph illustrating the change in skin lightness of a medium pigmented guinea pig following treatment with a deoxyArbutin composition described herein, after a period of twice-daily treatments, and after treatments were stopped.

FIG. 6 is a graph illustrating the change in skin lightness of a lighter pigmented guinea pig following treatment with a deoxyArbutin composition described herein, after a period of twice-daily treatments, and after treatments were stopped.

### DETAILED DESCRIPTION

Compositions that include effective skin-lightening agents are described herein. The compositions comprise a deoxyArbutin compound or a derivative thereof, as well as at least one of an antioxidant, a glycol, a solvent, and optional additional components. Such compositions may be prepared under an inert atmosphere to limit exposure to dioxygen, and may also involve careful monitoring to keep the pH within a specified range throughout the process. The resulting compositions may remain stable when subsequently placed under atmospheric conditions, and specifically may not substantially change in color.

The activity and potency of deoxyArbutin tyrosinase inhibitors is well-known and well-documented in the literature. See, for example: Hamed et al. J. Cosmet. Sci. 54(4) 2006 291-308; and Boissy et al. Experimental Dermatology 14 (8) 2005 601-608. It is safer and less irritating than the main skin-lightening active currently in use in the United States, hydroquinone, which belongs to the only other known class of reversible inhibitors that work topically. In many parts of the world, hydroquinone is banned from topical formulations due to safety concerns. Although those concerns have also been expressed by the US FDA, to date sales of hydroquinone are allowed in the United States (see, e.g., http://www.medicinenet.com/script/main/art.asp?articlekey=64167). There remains a need for a safer alternative to hydroquinone. Given its impressive in vivo activity and safety record, deoxyArbutin would be a natural replacement. However, all attempts to create a stable, non-browning topical formulation have been unsuccessful.

The literature teaches the instability of deoxyArbutin compounds in an aqueous environment, and commercial products, such as Prevage MD (Allergan) that contained deoxyArbutin, were withdrawn from the market, in part because the creams quickly and readily turned colored. Such browning can occur within hours of leaving the bottle, and is a significant barrier to commercial sales. The literature teaches the instability of deoxyArbutin compounds in an aqueous environment, and commercial products, such as Prevage MD (Allergan) that contained deoxyArbutin, were withdrawn from the market, in part because the creams quickly and readily turned colored. Such browning can occur within hours of leaving the bottle, and is a significant barrier to commercial sales.

Described herein are formulations, such as aqueous-based creams, that resists browning and remains stable, even when exposed to air at higher temperatures, for months rather than hours.

### DEFINITIONS

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

A “deoxyArbutin compound” refers to a compound that contains a mixed-ketal group, in which one oxygen atom of the mixed ketal group is substituted with a parahydroxyphenyl group (substituted or unsubstituted) and the other oxygen is substituted with a carbon ring or chain (e.g., a substituted or unsubstituted alkyl, heteroalkyl, alkenyl, alkynyl, aryl or heteroaryl group). As those skilled in the art will appreciate, a mixed-ketal group refers to a moiety in which a central carbon atom is attached via single bonds to two oxygen atoms. In a deoxyArbutin compound, the center carbon must have at least one of its two remaining valences substituted by a carbon ring or chain (e.g., a substituted or unsubstituted alkyl, heteroalkyl, alkenyl, alkynyl, aryl or heteroaryl group). Exemplary deoxyArbutin compounds include compounds of formulae I, II and III described herein. The term “deoxyArbutin” when used alone specifically refers to the compound 4-(tetrahydro-2H-pyran-2-yloxy)phenol. The terms “deoxyArbutin” and “deoxyArbutin compound” include deoxyArbutin and deoxyArbutin compounds in all isomeric forms (e.g., enantiomeric and diasteriomeric forms) and mixtures thereof. For example, deoxyArbutin refers to (R)-4-(tetrahydro-2H-pyran-2-yloxy)phenol, (S)-4-(tetrahydro-2H-pyran-2-yloxy)phenol, and mixtures thereof in any ratios.

“Chiral, non-racemic” is intended to encompass compounds that contain at least one chiral center, and do not have equal amounts of both enantiomers. It is contemplated explicitly herein, that the percent enantiomeric excess (% ee) of the more potent and more useful chiral form, will be from about 0.01% ee to about 100% ee. “Chiral, non-racemic” is intended to encompass compounds of the (+) as well as (−) optical activity, as each has utility independently, as well as in all admixtures as described above.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

“Alkyl” refers to a saturated aliphatic hydrocarbon including straight chain and branched chain groups. “Alkyl” may be exemplified by groups such as methyl, ethyl, n-propyl, isopropyl, n-butyl and the like. Alkyl groups may be substituted or unsubstituted. Substituents may also be themselves substituted. When substituted, the substituent group may be (but is not limited to) C₁-C₄ alkyl, aryl, amino, cyano, halogen, alkoxy or hydroxyl. “C₁-C₄ alkyl” refers to alkyl groups containing one to four carbon atoms.

“Alkenyl” refers to an unsaturated aliphatic hydrocarbon moiety including straight chain and branched chain groups. Alkenyl moieties must contain at least one alkene. “Alkenyl” may be exemplified by groups such as ethenyl, n-propenyl, isopropenyl, n-butenyl and the like. Alkenyl groups may be substituted or unsubstituted. Substituents may also be themselves substituted. When substituted, the substituent group may be, e.g., alkyl, halogen or alkoxy. Substituents may also be themselves substituted. Substituents be placed on the alkene itself and also on the adjacent member atoms or the alkynyl moiety “C₂-C₄ alkenyl” refers to alkenyl groups containing two to four carbon atoms.

“Alkynyl” refers to an unsaturated aliphatic hydrocarbon moiety including straight chain and branched chain groups. Alkynyl moieties must contain at least one alkyne. “Alkynyl” may be exemplified by groups such as ethynyl, propynyl, n-butynyl and the like. Alkynyl groups may be substituted or unsubstituted. When substituted, the substituent group may be, e.g., alkyl, amino, cyano, halogen, alkoxyl or hydroxyl. Substituents may also be themselves substituted. Substituents are not on the alkyne itself but on the adjacent member atoms of the alkynyl moiety. “C₂-C₄ alkynyl” refers to alkynyl groups containing two to four carbon atoms.

“Acyl” or “carbonyl” refers to the group —C(O)R wherein R is alkyl; alkenyl; alkyl alkynyl, aryl, heteroaryl, carbocyclic, heterocarbocyclic; C₁-C₄ alkyl aryl or C₁-C₄ alkyl heteroaryl. C₁-C₄ alkylcarbonyl refers to a group wherein the carbonyl moiety is preceded by an alkyl chain of 1-4 carbon atoms.

“Alkoxy” refers to the group —O—R wherein R is acyl, alkyl alkenyl, alkyl alkynyl, aryl, carbocyclic; heterocarbocyclic; heteroaryl, C₁-C₄ alkyl aryl or C₁-C₄ alkyl heteroaryl

“Amino” refers to the group —NR′R′ wherein each R′ is, independently, hydrogen, alkyl, aryl, heteroaryl, C₁-C₄ alkyl aryl or C₁-C₄ alkyl heteroaryl. The two R′ groups may themselves be linked to form a ring.

“Aryl” refers to an aromatic carbocyclic group. “Aryl” may be exemplified by phenyl. The aryl group may be substituted or unsubstituted. Substituents may also be themselves substituted. When substituted, the substituent group is may be, e.g., heteroaryl, acyl, carboxyl, carbonylamino, nitro, amino, cyano, halogen, or hydroxyl.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.”

The term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

“Carboxyl” refers to the group —C(═O)O—C₁-C₄ alkyl.

“Carbonylamino” refers to the group —C(O)NR′R′ wherein each R′ is, independently, hydrogen, alkyl, aryl, cycloalkyl; heterocycloalkyl; heteroaryl, C₁-C₄ alkyl aryl or C₁-C₄ alkyl heteroaryl. The two R′ groups may themselves be linked to form a ring.

“C₁-C₄ alkyl aryl” refers to C₁-C₄ alkyl groups having an aryl substituent such that the aryl substituent is bonded through an alkyl group. “C₁-C₄ alkyl aryl” may be exemplified by benzyl.

“C₁-C₄ alkyl heteroaryl” refers to C₁-C₄ alkyl groups having a heteroaryl substituent such that the heteroaryl substituent is bonded through an alkyl group.

“Carbocyclic group” or “cycloalkyl” refers to a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, such as 4 to 7 carbon atoms or 5 to 6 carbon atoms in the ring. Bicyclic carbocyclic groups contain 8 to 12 carbon atoms, such as 9 to 10 carbon atoms in the ring. Carbocyclic groups may be substituted or unsubstituted. Substituents may also be themselves substituted. Carbocyclic groups include but are not limited to cyclopropyl, cyclobutyl, cyclopentyl, cyclohexyl, cyclohexenyl, and cycloheptyl. Suitable carbocyclic groups include cyclopropyl and cyclobutyl. Carbocyclic groups are not aromatic.

“Halogen” refers to fluoro, chloro, bromo or iodo moieties. Suitably, the halogen is fluoro, chloro, or bromo.

“Heteroaryl” or “heteroaromatic” refers to a monocyclic or bicyclic aromatic carbocyclic radical having one or more heteroatoms in the carbocyclic ring. Heteroaryl may be substituted or unsubstituted. When substituted, the substituents may themselves be substituted. Substituents include but are not limited to aryl; C₁-C₄ alkylaryl; amino; halogen, hydroxy, cyano, nitro; carboxyl; carbonylamino or C₁-C₄ alkyl. Suitable heteroaromatic groups include tetrazoyl, triazolyl; thienyl, thiazolyl, purinyl, pyrimidyl, pyridyl, furanyl, benzothiofuranyl, thienyl, furanyl, tetrazoyl, triazolyl and pyridyl.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

“Heteroalkyl” refers to an alkyl group in which at least one carbon atom is replaced with a heteroatom. An exemplary heteroalkyl group is a methoxymethyl group.

“Heteroatom” refers to an atom other than carbon in the ring of a heterocyclic group or a heteroaromatic group or the chain of a heteroalkyl group. Suitably, heteroatoms are selected from the group consisting of nitrogen, sulfur, and oxygen atoms. Groups containing more than one heteroatom may contain the same or different heteroatoms.

“Heterocarbocyclic group” or “heterocycloalkyl” or “heterocyclic” means a monovalent saturated or partially unsaturated hydrocarbon ring containing at least one heteroatom. Heterocarbocyclic groups are monocyclic, or are fused, spiro, or bridged bicyclic ring systems. Monocyclic heterocarbocyclic groups contain 3 to 10 carbon atoms, suitably 4 to 7 carbon atoms or 5 to 6 carbon atoms in the ring. Bicyclic heterocarbocyclic groups contain 8 to 12 carbon atoms, e.g., 9 to 10 carbon atoms in the ring. Heterocarbocyclic groups may be substituted or unsubstituted. Substituents may also be themselves substituted. Exemplary heterocarbocyclic groups include epoxy, tetrahydrofuranyl, azacyclopentyl, azacyclohexyl, piperidyl, homopiperidyl, piperidyl, and homopiperidyl. A suitable heterocarbocyclic group is piperidyl.

“Hydroxy” or “hydroxyl” refers to —OH. Alcohols contain hydroxy groups. Hydroxy groups may be free or protected.

“Linker” means a linear chain of n member atoms where n is an integer of from 1 to 4.

“Member atom” means a carbon, nitrogen, oxygen or sulfur atom. Member atoms may be substituted up to their normal valence. If substitution is not specified the substituents required for valency are hydrogen.

“Ring” means a collection of member atoms that are cyclic. Rings may be carbocyclic, aromatic, or heterocyclic or heteroaromatic, and may be substituted or unsubstituted, and may be saturated or unsaturated. Ring junctions with the main chain may be fused or spirocyclic. Rings may be monocyclic or bicyclic. Rings contain at least 3 member atoms and at most 10 member atoms. Monocyclic rings may contain 3 to 7 member atoms and bicyclic rings may contain from 8 to 12 member atoms. Bicyclic rings themselves may be fused or spirocyclic.

“Thioalkyl” refers to the group —S-alkyl.

“Sulfonyl” refers to the —S(O)₂R′ group wherein R′ is alkoxy, alkyl, aryl, carbocyclic, heterocarbocyclic; heteroaryl, C₁-C₄ alkyl aryl or C₁-C₄ alkyl heteroaryl.

“Sulfonylamino” refers to the —S(O)₂NR′R′ group wherein each R′ is independently alkyl, aryl, heteroaryl, C₁-C₄ alkyl aryl or C₁-C₄ alkyl heteroaryl.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, —(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

“Pharmaceutically or cosmetically acceptable carrier” means a carrier that is useful for the preparation of a pharmaceutical composition that is at least one of: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal and oral administration. “Pharmaceutically or cosmetically acceptable carrier” also includes agents for preparation of aqueous dispersions and sterile powders for injection or dispersions.

“Excipient” includes physiologically compatible additives useful in preparation of a pharmaceutical composition. Examples of pharmaceutically or cosmetically acceptable carriers and excipients can for example be found in Remington Pharmaceutical Science, 16th Ed.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

“Substantially free,” as used herein in the context of a component of a composition, means that the composition is completely free of the indicated component, or includes only a trace amount of the indicated component. A “trace amount” may be less than 1% by weight, less than 0.5% by weight, or less than 0.1% by weight.

“Therapeutically effective amount” as used herein refers to a dosage of the compounds or compositions effective for influencing, reducing or inhibiting the activity of or preventing activation melanocytes. This term as used herein may also refer to an amount effective at bringing about a desired in vivo effect in an animal (e.g., a human), such as reduction in overall pigmentation, or a local reduction in pigmentation.

“Administering” refers to administration of the compounds as needed to achieve the desired effect.

The term “disease or condition associated with melanin formation” is used to mean a disease or condition treatable, in whole or in part, by inhibition of pigment formation.

The term “controlling the disease or condition” is used to mean changing the activity of one or more enzymes to affect the disease or condition.

It is specifically understood that any numerical value recited herein (e.g., ranges) includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. These are only examples of what is specifically intended. With respect to amounts of components, all percentages are by weight, unless explicitly indicated otherwise.

### Compounds

Compounds that may be used in the compositions described herein include deoxyArbutin compounds. Such compounds may alter the amount of pigment produced by the skin, and may accordingly convey beautification benefits to human skin. Use of such compounds may also discourage the onset of skin disorders. Without wishing to be bound by theory, it is believed that the ability of the present compounds to discourage skin pigmentation is due, at least in part, to the compounds' ability to inhibit enzymes such as tyrosinase, as well as their abilities to resist oxidation by those same enzymes.

Compounds that may be used in the compositions of the present invention include compounds of formula I:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur.

In certain embodiments, the Z group of formula I is O. In some embodiments, the Z group of formula I is NR₁. In some embodiments, the Z group of formula I is NH. In other embodiments, the Z group of formula I is SO. In some embodiments, the Z group of formula I is SO₂.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In certain embodiments, each of A and B is independently an optionally substituted group selected from C1-6 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, or sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur.

In some embodiments, A and B of formula I are taken together with the atoms to which they are attached to form an optionally substituted saturated or partially unsaturated ring having from 4-9 member atoms and 0-2 heteroatoms independently selected from nitrogen, oxygen, or sulfur.

In some embodiments, A and B of formula I are taken together to form a 5-8 member monocyclic ring having 0-2 heteroatoms independently selected from nitrogen, oxygen, and sulfur. In some embodiments, A and B of formula I are taken together to form a 5-6 member monocyclic ring having 0-1 heteroatoms independently selected from nitrogen, oxygen, and sulfur. In some embodiments, A and B of formula I are taken together to form a 5 member monocyclic ring. In some embodiments, A and B of formula I are taken together to form a 6 member monocyclic ring. Exemplary monocyclic rings formed by A and B are depicted in table of representative examples of chiral non-racemic compounds, below.

In certain embodiments, A and B of formula I are taken together to form an 8-12 member bicyclic ring having 0-2 heteroatoms independently selected from nitrogen, oxygen, and sulfur. In some embodiments, A and B of formula I are taken together to form 10 member bicyclic ring having 0-2 heteroatoms. Exemplary bicyclic rings formed by A and B are depicted in table of representative examples of compounds, below.

In some embodiments, compounds that may be used in the compositions of the present invention include compounds of formula I-a:

or a pharmaceutically acceptable salt thereof, wherein Z, X₁ and X₂ are as defined and described above and herein, n is 1, 2, 3, 4 or 5, each R is independently selected from hydrogen and C1-6 aliphatic, and each R′ is independently selected from hydrogen, C1-6 aliphatic and —OR^{a}, wherein each R^{a} is independently selected from hydrogen and C1-6 aliphatic.

In some embodiments, n is 2, 3 or 4. In some embodiments, n is 2 or 3. In some embodiments, each occurrence of R and R′ is hydrogen. In some embodiments, at least one occurrence of R′ is —OR^{a}, wherein R^{a} is hydrogen or C1-6 aliphatic (e.g., methyl). In some embodiments, In some embodiments, Z is O. In some embodiments, Z is S. In some embodiments, Z is SO. In some embodiments, Z is SO₂. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, compounds that may be used in the compositions of the present invention include compounds of formula I-b:

or a pharmaceutically acceptable salt thereof, wherein Z, X₁ and X₂ are as defined and described above and herein.

In some embodiments, Z is O. In some embodiments, Z is S. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, compounds that may be used in the compositions of the present invention include compounds of formula I-c:

or a pharmaceutically acceptable salt thereof, wherein X₁ and X₂ are as defined and described above and herein.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, compounds that may be used in the compositions of the present invention include compounds of formula I-d:

or a pharmaceutically acceptable salt thereof, wherein Z, X₁ and X₂ are as defined and described above and herein, and R^{a} and R^{b} are each independently selected from the group consisting of hydrogen, optionally substituted C1-6 aliphatic, and —OR², wherein R² is selected from the group consisting of hydrogen and optionally substituted C1-6 aliphatic.

In some embodiments, Z is O. In some embodiments, Z is S. In some embodiments, Z is SO. In some embodiments, Z is SO₂. In some embodiments, each of R^{a} and R^{b} is hydrogen. In some embodiments, R^{a} is optionally substituted C1-6 aliphatic, e.g., methyl or —CH₂OH. In some embodiments, R^{b} is —OR² (e.g., —OH). In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, compounds that may be used in the compositions of the present invention include compounds of formula I-e:

or a pharmaceutically acceptable salt thereof, wherein X₁ and X₂ are as defined and described above and herein.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, compounds that may be used in the compositions of the present invention include compounds of formula I-f:

or a pharmaceutically acceptable salt thereof, wherein:
Z is selected from NR₁, S, O, SO and SO₂;
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from the group consisting of: C1-6 aliphatic; a 3-8 membered saturated carbocyclic ring; and phenyl.

In some embodiments, Z is O. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is nitro. In some embodiments, one of X₁ and X₂ is hydrogen and the other is C1-6 aliphatic. In some embodiments, one of X₁ and X₂ is hydrogen and the other is methyl. In some embodiments, A is C1-6 aliphatic. In some embodiments, A is methyl or ethyl. In some embodiments, A is a 3-8 membered saturated carbocyclic ring. In some embodiments, A is cyclohexyl. In some embodiments, A is phenyl. In some embodiments, B is C1-6 aliphatic. In some embodiments, B is ethyl or n-propyl. In some embodiments, B is a 3-8 membered saturated carbocyclic ring. In some embodiments, B is cyclopentyl.

Exemplary compounds of formula I include but are not limited to the following:

A suitable deoxyArbutin compound is deoxyArbutin (4-(tetrahydro-2H-pyran-2-yloxy)phenol).

Certain compounds may exist in one or more particular geometric, optical, enantiomeric, diastereomeric, epimeric, stereoisomeric, tautomeric, conformational, or anomeric forms, including but not limited to, cis- and trans-forms; E- and Z-forms; c-, t-, and r-forms; endo and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”). Unless otherwise specified, a reference to a particular compound includes all such isomeric forms, including (wholly or partially) racemic and other mixtures thereof. Methods for the preparation (e.g. asymmetric synthesis) and separation (e.g., fractional crystallization and chromatographic means) of such isomeric forms are either known in the art or are readily obtained using methods described herein.

Chiral, Non-Racemic Compounds

Certain chiral, non-racemic compounds may also be used in the present compositions. In some embodiments, a compound that may be used in the compositions of the present invention include a compound of formula II or formula III, as described in detail below and herein, that is enantiomerically enriched. As used herein, the term “enantiomerically enriched”, as used herein signifies that one enantiomer makes up at least 80% or 85% of the preparation. In certain embodiments, the term enantiomerically enriched signifies that at least 90% of the preparation is one of the enantiomers. In other embodiments, the term signifies that at least 95% of the preparation is one of the enantiomers.

In certain embodiments, the composition of the present invention comprises a compound of formula II having a % enantiomeric excess (% ee) of at least 50%. In certain embodiments, the composition of the present invention comprises a compound of formula II having a % enantiomeric excess (% ee) of at least 60%. In certain embodiments, the composition of the present invention comprises a compound of formula II having a enantiomeric excess (% ee) of at least 70%. In certain embodiments, the composition of the present invention comprises a compound of formula II having a % enantiomeric excess (% ee) of at least 80%. In certain embodiments, the composition of the present invention comprises a compound of formula II having a % enantiomeric excess (% ee) of at least 90%. In some embodiments, the composition of the present invention comprises a compound of formula II having a % ee of at least 95%. In some embodiments, the composition of the present invention comprises a compound of formula II having a % ee of at least 98%. In some embodiments, the composition of the present invention comprises a compound of formula II having a % ee of at least 99%.

In certain embodiments, the composition of the present invention comprises a compound of formula III having a % enantiomeric excess (% ee) of at least 50%. In certain embodiments, the composition of the present invention comprises a compound of formula III having a % enantiomeric excess (% ee) of at least 60%. In certain embodiments, the composition of the present invention comprises a compound of formula III having a enantiomeric excess (% ee) of at least 70%. In certain embodiments, the composition of the present invention comprises a compound of formula III having a % enantiomeric excess (% ee) of at least 80%. In certain embodiments, the composition of the present invention comprises a compound of formula III having a % enantiomeric excess (% ee) of at least 90%. In some embodiments, the composition of the present invention comprises a compound of formula III having a % ee of at least 95%. In some embodiments, the composition of the present invention comprises a compound of formula III having a % ee of at least 98%. In some embodiments, the composition of the present invention comprises a compound of formula III having a % ee of at least 99%.

In certain embodiments, the composition of the present invention comprises a compound of formula II, as defined and described herein, substantially free of a compound of formula III.

In certain embodiments, the composition of the present invention comprises a compound of formula III, as defined and described herein, substantially free of a compound of formula II.

“Substantially free,” as used herein in the context of enantiomers, means that the compound is made up of a significantly greater proportion of one enantiomer. In other embodiments, at least about 95% by weight of a desired enantiomer is present. In still other embodiments of the invention, at least about 99% by weight of a desired enantiomer is present. Such enantiomers may be isolated from racemic mixtures by any method known to those skilled in the art, including high performance liquid chromatography (HPLC).

In embodiments, chiral, non-racemic compounds illustrated by the following general structures formula II and formula III may be used in the compositions described herein:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur.

Note that, other than in the chiral sense, both structures are identical, but they are drawn to specifically exemplify both enantiomers.

In some embodiments, the composition of the present invention comprises a compound of formula II:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C₁₋₁₀ aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur.

In some embodiments, the composition of the present invention a compound of formula III:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur.

In certain embodiments, the Z group of formula II or III is O. In some embodiments, the Z group of formula II or III is NR₁. In some embodiments, the Z group of formula II or III is NH. In other embodiments, the Z group of formula II or III is SO. In some embodiments, the Z group of formula II or III is SO₂.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In certain embodiments, the composition of the present invention comprises a compound of either of formula II or III, wherein each of A and B is independently an optionally substituted group selected from C1-6 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, or sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur.

In some embodiments, the composition of the present invention comprises a compound of either of formula II or III wherein A and B are taken together with the atoms to which they are attached to form an optionally substituted saturated or partially unsaturated ring having from 4-9 member atoms and 0-2 heteroatoms independently selected from nitrogen, oxygen, or sulfur.

In some embodiments, A and B of formula II or III are taken together to form a 5-8 member monocyclic ring having 0-2 heteroatoms independently selected from nitrogen, oxygen, and sulfur. In some embodiments, A and B of formula II or III are taken together to form a 5-6 member monocyclic ring having 0-1 heteroatoms independently selected from nitrogen, oxygen, and sulfur. In some embodiments, A and B of formula II or III are taken together to form a 5 member monocyclic ring. In some embodiments, A and B of formula II or III are taken together to form a 6 member monocyclic ring. Exemplary monocyclic rings formed by A and B are depicted in table of representative examples of chiral non-racemic compounds, below.

In certain embodiments, A and B of formula II or III are taken together to form an 8-12 member bicyclic ring having 0-2 heteroatoms independently selected from nitrogen, oxygen, and sulfur. In some embodiments, A and B of formula II or III are taken together to form 10 member bicyclic ring having 0-2 heteroatoms. Exemplary bicyclic rings formed by A and B are depicted in table of representative examples of chiral non-racemic compounds, below.

In some embodiments, the composition of the present invention comprises a compound of formula I-a:

or a pharmaceutically acceptable salt thereof, wherein Z, X₁ and X₂ are as defined and described above and herein, n is 1, 2, 3, 4 or 5, each R is independently selected from hydrogen and C1-6 aliphatic, and each R′ is independently selected from hydrogen, C1-6 aliphatic and —OR′, wherein each R^{a} is independently selected from hydrogen and C1-6 aliphatic.

In some embodiments, n is 2, 3 or 4. In some embodiments, n is 2 or 3. In some embodiments, each occurrence of R and R′ is hydrogen. In some embodiments, at least one occurrence of R′ is —OR^{a}, wherein R^{a} is hydrogen or C1-6 aliphatic (e.g., methyl). In some embodiments, In some embodiments, Z is O. In some embodiments, Z is S. In some embodiments, Z is SO. In some embodiments, Z is SO₂. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula II-a, substantially free of a compound of formula III-a.

In some embodiments, the composition of the present invention comprises a compound of formula II-b:

or a pharmaceutically acceptable salt thereof, wherein Z, X₁ and X₂ are as defined and described above and herein.

In some embodiments, Z is O. In some embodiments, Z is S. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula II-b, substantially free of a compound of formula III-b.

In some embodiments, the composition of the present invention comprises a compound of formula II-c:

or a pharmaceutically acceptable salt thereof, wherein X₁ and X₂ are as defined and described above and herein.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula II-c, substantially free of a compound of formula III-c.

In some embodiments, the composition of the present invention comprises a compound of formula II-d:

or a pharmaceutically acceptable salt thereof, wherein Z, X₁ and X₂ are as defined and described above and herein, and R^{a} and R^{b} are each independently selected from the group consisting of hydrogen, optionally substituted C1-6 aliphatic, and —OR², wherein R² is selected from the group consisting of hydrogen and optionally substituted C1-6 aliphatic.

In some embodiments, Z is O. In some embodiments, Z is S. In some embodiments, Z is SO. In some embodiments, Z is SO₂. In some embodiments, each of R^{a} and R^{b} is hydrogen. In some embodiments, R^{a} is optionally substituted C1-6 aliphatic, e.g., methyl or —CH₂OH. In some embodiments, R^{b} is —OR² (e.g., —OH). In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula II-d, substantially free of a compound of formula III-d.

In some embodiments, the composition of the present invention comprises a compound of formula II-e:

or a pharmaceutically acceptable salt thereof, wherein X₁ and X₂ are as defined and described above and herein.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula II-e, substantially free of a compound of formula III-e.

In some embodiments, the composition of the present invention comprises a compound of formula II-f:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², SR², N(R²)₂, —COOR², and —CON(R²)₂;
each R² is independently hydrogen or an optionally substituted C1-6 aliphatic; and
A and B are independently an optionally substituted group selected from the group consisting of: C1-6 aliphatic; a 3-8 membered saturated carbocyclic ring; and phenyl.

In some embodiments, Z is O. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is nitro. In some embodiments, one of X₁ and X₂ is hydrogen and the other is C1-6 aliphatic. In some embodiments, one of X₁ and X₂ is hydrogen and the other is methyl. In some embodiments, A is C1-6 aliphatic. In some embodiments, A is methyl or ethyl. In some embodiments, A is a 3-8 membered saturated carbocyclic ring. In some embodiments, A is cyclohexyl. In some embodiments, A is phenyl. In some embodiments, B is C1-6 aliphatic. In some embodiments, B is ethyl or n-propyl. In some embodiments, B is a 3-8 membered saturated carbocyclic ring. In some embodiments, B is cyclopentyl.

In some embodiments, the composition of the present invention comprises a compound of formula II-f, substantially free of a compound of formula III-f.

In some embodiments, the composition of the present invention comprises a compound of formula III-a:

or a pharmaceutically acceptable salt thereof, wherein Z, X₁ and X₂ are as defined and described above and herein, n is 1, 2, 3, 4 or 5, each R is independently selected from hydrogen and C1-6 aliphatic, and each R′ is independently selected from hydrogen, C1-6 aliphatic and —OR^{a}, wherein each R^{a} is independently selected from hydrogen and C1-6 aliphatic.

In some embodiments, n is 2, 3 or 4. In some embodiments, n is 2 or 3. In some embodiments, each occurrence of R and R′ is hydrogen. In some embodiments, at least one occurrence of R′ is —OR^{a}, wherein R^{a} is hydrogen or C1-6 aliphatic (e.g., methyl). In some embodiments, In some embodiments, Z is O. In some embodiments, Z is S. In some embodiments, Z is SO. In some embodiments, Z is SO₂. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula III-a, substantially free of a compound of formula II-a.

In some embodiments, the composition of the present invention comprises a compound of formula III-b:

or a pharmaceutically acceptable salt thereof, wherein Z, X₁ and X₂ are as defined and described above and herein.

In some embodiments, Z is O. In some embodiments, Z is S. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula III-b, substantially free of a compound of formula II-b.

In some embodiments, the composition of the present invention comprises a compound of formula III-c:

or a pharmaceutically acceptable salt thereof, wherein X₁ and X₂ are as defined and described above and herein.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula III-c, substantially free of a compound of formula II-c.

In some embodiments, the composition of the present invention comprises a compound of formula III-d:

or a pharmaceutically acceptable salt thereof, wherein Z, X₁ and X₂ are as defined and described above and herein, and R^{a} and R^{b} are each independently selected from the group consisting of hydrogen, optionally substituted C1-6 aliphatic, and —OR², wherein R² is selected from the group consisting of hydrogen and optionally substituted C1-6 aliphatic.

In some embodiments, Z is O. In some embodiments, Z is S. In some embodiments, Z is SO. In some embodiments, Z is SO₂. In some embodiments, each of R^{a} and R^{b} is hydrogen. In some embodiments, R^{a} is optionally substituted C1-6 aliphatic, e.g., methyl or —CH₂OH. In some embodiments, R^{b} is —OR² (e.g., —OH). In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula III-d, substantially free of a compound of formula II-d.

In some embodiments, the composition of the present invention comprises a compound of formula III-e:

or a pharmaceutically acceptable salt thereof, wherein X₁ and X₂ are as defined and described above and herein.

In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is C1-6 aliphatic. In some embodiments, one of X₁ and X₂ is hydrogen and the other is methyl.

In some embodiments, the composition of the present invention comprises a compound of formula III-e, substantially free of a compound of formula II-e.

In some embodiments, the composition of the present invention comprises a compound of formula III-f:

or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from the group consisting of: C1-6 aliphatic; a 3-8 membered saturated carbocyclic ring; and phenyl.

In some embodiments, Z is O. In some embodiments, each of X₁ and X₂ is hydrogen. In other embodiments, one of X₁ and X₂ is hydrogen and the other is halogen, cyano, nitro, optionally substituted C1-6 aliphatic, —OR², —SR², —N(R²)₂, —COOR², and —CON(R²)₂. In some embodiments, one of X₁ and X₂ is hydrogen and the other is halogen. In some embodiments, one of X₁ and X₂ is hydrogen and the other is fluoro or chloro. In some embodiments, one of X₁ and X₂ is hydrogen and the other is nitro. In some embodiments, one of X₁ and X₂ is hydrogen and the other is C1-6 aliphatic. In some embodiments, one of X₁ and X₂ is hydrogen and the other is methyl. In some embodiments, A is C1-6 aliphatic. In some embodiments, A is methyl or ethyl. In some embodiments, A is a 3-8 membered saturated carbocyclic ring. In some embodiments, A is cyclohexyl. In some embodiments, A is phenyl. In some embodiments, B is C1-6 aliphatic. In some embodiments, B is ethyl or n-propyl. In some embodiments, B is a 3-8 membered saturated carbocyclic ring. In some embodiments, B is cyclopentyl.

In some embodiments, the present invention provides a compound of formula III-f, substantially free of a compound of formula II-f.

Exemplary compounds of formula II and formula III include but are not limited to the following:

### Examples

### Cyclic deoxyArbutins

```
  (S)-4-(tetrahydro-2H- pyran-2-yloxy)phenol
  (R)-4-(tetrahydro-2H- pyran-2-yloxy)phenol
  (S)-2-chloro-4-(tetrahydro- 2H-pyran-2-yloxy)phenol
  (R)-2-fluoro-4- (tetrahydro-2H-pyran-2- yloxy)phenol
  (S)-2-fluoro-4-(tetrahydro- 2H-pyran-2-yloxy)phenol
  (R)-2-chloro-4-(tetrahydro- 2H-pyran-2-yloxy)phenol
  (S)-2-methoxy-4-(tetrahydro- 2H-pyran-2-yloxy)phenol
  (R)-2-methoxy-4-(tetrahydro- 2H-pyran-2-yloxy)phenol
  (R)-2-hydroxy-5-(tetrahydro-2H- pyran-2-yloxy)benzonitrile
  (S)-2,5-difluoro-4-(tetrahydro- 2H-pyran-2-yloxy)phenol
  4-((2R,3S)-3-methoxytetrahydro-2H- pyran-2-yloxy)phenol
  2-fluoro-4-((2S,3R)-3- methoxytetrahydro-2H-pyran-2- yloxy)phenol
```

### Examples

### Other Ring Sizes

```
  (S)-2-fluoro-4-(tetrahydrofuran-2- yloxy)phenol
  (R)-2-fluoro-4-(tetrahydrofuran- 2-yloxy)phenol
  (S)-2-fluoro-4- (tetrahydrofuran-2- yloxy)phenol
  2-fluoro-4-((2S,5S)-2- methyltetrahydrofuran-2- yloxy)phenol
  2-fluoro-4-((2S,5R)-5- (hydroxymethyl)tetrahydrofuran- 2-yloxy)phenol
  4-((2S,5S)-5- (hydroxymethyl)tetrahydrofuran- 2-yloxy)-2-methylphenol
  (2S,3R)-2-(4-hydroxy-3- methylphenoxy)tetrahydrofuran- 3-ol
  (S)-2-chloro-4-(oxepan-2- yloxy)phenol
  (R)-2-fluoro-4-(oxepan-2- yloxy)phenol
  2-fluoro-4-((2S)- octahydrocyclopenta[b]pyran-2- yloxy)phenol
  2-fluoro-4-((6R)-hexahydro-2H- furo[2,3-b]pyran-6-yloxy)phenol
  (R)-4-(1,4-dioxan-2-yloxy)-2- fluorophenol
  (R)-2-fluoro-4-(oxocan- 2-yloxy)phenol
  (S)-2-fluoro-4-(oxocan- 2-yloxy)phenol
  (S)-4-(chroman-2-yloxy)-2- iodophenol
  (R)-2-fluoro-4-(1,2,3,4- tetrahydroquinolin-2-yloxy)phenol
```

### Examples

### ThiodeoxyArbutins

```
  (S)-4-(tetrahydro-2H-thiopyran-2- yloxy)phenol
  (R)-4-(tetrahydro-2H-thiopyran-2- yloxy)phenol
  (R)-3-fluoro-4-(tetrahydro-2H- thiopyran-2-yloxy)phenol
  (S)-3-fluoro-4-(tetrahydro-2H- thiopyran-2-yloxy)phenol
  (R)-2,3-difluoro-4-(tetrahydro-2H- thiopyran-2-yloxy)phenol
  (R)-2,4-difluoro-4-(tetrahydro-2H- thiopyran-2-yloxy)phenol
  2-fluoro-4-((2R,3R)-3- methoxytetrahydro-2H-thiopyran-2- yloxy)phenol
```

### Examples

### Other Thio Analogs

```
  (R)-2-fluoro-4-(tetrahydrothiophen- 2-yloxy)phenol
  (S)-2-fluoro-4-(tetrahydrothiophen-2- yloxy)phenol
  (S)-4-(2,3-dihydrobenzo[b]thiophen- 2-yloxy)phenol



```

### Examples

### Brominated Analogs

```
  (R)-2-bromo-4-(tetrahydro- 2H-pyran-2-yloxy)phenol
  2-bromo-4-((2R,6R)-6- methyltetrahydro-2H-pyran- 2-yloxy)phenol
  (S)-2-bromo-4-(tetrahydrofuran-2- yloxy)phenol
```

### Examples

### Acyclic Analogs

```
  (S)-4-(1-methoxypropoxy)phenol
  (R)-4-(1-methoxypropoxy)phenol
  (R)-4-(1-ethoxypropoxy)phenol
  (S)-4-(1-ethoxybutoxy)phenol
  (R)-4-(1-ethoxybutoxy)-2- fluorophenol
  (R)-5-(1-ethoxypropoxy)-2- hydroxybenzonitrile
```

### Examples

### Other Acyclic Analogs

```
  (S)-2-chloro-4-(1- (cyclohexyloxy)propoxy)phenol
  (S)-2-fluoro-4-(1- phenoxypropoxy)phenol
  (R)-4- (cyclopentyl(ethoxy)methoxy)-2- fluorophenol
```

Chiral, non-racemic compounds are described in a provisional patent application entitled “CHIRAL COMPOUNDS, COMPOSITIONS, PRODUCTS AND METHODS EMPLOYING SAME” filed on Jan. 5, 2012 (attorney docket no. 029639-9002-US00).

Salts, Isomers, Protected Forms, and Prodrugs

Unless otherwise specified, a reference to a particular compound also includes ionic, salt, solvate, and protected forms of thereof, for example, as discussed below. It may be convenient or desirable to prepare, purify, and/or handle a corresponding salt of the active compound, for example, a pharmaceutically-acceptable salt. Examples of pharmaceutically acceptable salts are discussed in Berge et al., J. Pharm. Sci., 66, 1-19 (1977). Exemplary pharmaceutically acceptable salts include hydrochloride salts.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and paramethoxyphenyl).

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. For example, an amine group may be protected, for example, as an amide or a urethane, for example, as: a methyl amide (—NHCO—CH3); a benzyloxy amide (—NHCO—OCH₂C₆H₅, —NHCbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide.

For example, a carboxylic acid group may be protected as an ester for example, as: an C₁₋₇ alkyl ester (e.g. a methyl ester; a t-butyl ester); a C₁₋₇haloalkyl ester (e.g., a C₁₋₇ trihaloalkylester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃). It may be convenient or desirable to prepare, purify, and/or handle the active compound in the form of a prodrug.

The term “prodrug”, as used herein, pertains to a compound which, when metabolized (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₇ alkyl (e.g. -Me, -Et); C₁₋₇ aminoalkyl (e g aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexylcarbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Combinations of deoxyArbutins

Compounds may be employed individually or in combination. In combination therapies, deoxyArbutin compounds such as those described herein may exhibit heightened performance and synergy in the beautification of mammalian skin.

Combinations which may be particularly effective are shown in the table below. Each composition includes two components; component A is the (−) enantiomer of deoxyArbutin (4-(tetrahydro-2H-pyran-2-yloxy)phenol), while component B is a second deoxyArbutin compound as indicated below.

| Composition | Component A | Component B |
|---|---|---|
| Alpha-1 | 0.1-5% (−) deoxyArbutin | 0.1-3% (−) 2-fluoro |
| | | deoxyArbutin |
| | | (2-fluoro-4-(tetrahydro-2H- |
| | | pyran-2-yloxy)phenol) |
| Beta-1 | 0.1-5% (−) deoxyArbutin | 0.1-3% (−) 2-chloro |
| | | deoxyArbutin |
| | | (2-chloro-4-(tetrahydro-2H- |
| | | pyran-2-yloxy)phenol) |
| Gamma-1 | 0.1-5% (−) deoxyArbutin | 0.1-3% (−) 2,5-dichloro |
| | | deoxyArbutin |
| | | (2,5-dichloro-4-(tetrahydro- |
| | | 2H-pyran-2-yloxy)phenol) |
| Delta-1 | 0.1-5% (−) deoxyArbutin | 0.1-3% thio-deoxyArbutin |
| | | (4-(tetrahydro-2H- |
| | | thiopyran-2-yloxy)phenol) |
| Alpha-2 | 0.1-5% (−) deoxyArbutin | 0.1-5% 2-fluoro-thio- |
| | | deoxyArbutin |
| | | (2-fluoro-4-(tetrahydro-2H- |
| | | thiopyran-2-yloxy)phenol) |

Methods of Separating (−) from (+) Enantiomers

Many methods of chiral separation exist in the art, yet none have been applied to the separation of compounds that have a single chiral center in an acetal or ketal linkage, and no other chiral moiety. For example, there are many examples of molecules containing a tetrahydropyranyl moiety, but no examples where, when the chiral center of the tetrahydropyran is the only chiral center, a chiral separation has been successfully performed. Indeed, the failure of the ChiralPak IA, 4.6×250 mm (Diacel Chemical Ind., Ltd.) method of separation teaches that such compounds are not amenable to separation, and the temporal nature of the THP group; its main use as a temporary, or protecting group, has resulted in either a lack of interest or lack of progress in this area. For example, the art describes the reaction of (−) and (+) glycidol with dihydropyran in WO2010027113A2: Process for Preparing (S)-(−)-Felodipine, but the resulting tetrahydropyran is not the object of the separation, nor does it materially participate in the chemistry. U.S. Pat. No. 7,393,858 describes tetrahydropyran compounds as tachykinin antagonists, but here again the chiral center of the instant disclosure is not the method by which the enantiomers are separated.

Methods of Synthesizing/Purifying deoxyArbutin Compounds

DeoxyArbutin compounds (e.g., compounds of formulae I, II and III) can be prepared using methods known in the art. For example, the compounds can be prepared using methods described in U.S. Pat. Nos. 6,068,834 and 6,537,527, which are hereby incorporated by reference in their entireties.

It may be useful to further purify the deoxyArbutin compound prior to incorporation in to a composition, such as a composition described herein. Accordingly, in some embodiments, a deoxyArbutin compound may be recrystallized from an appropriate solvent system. For example, a deoxyArbutin compound may be recrystallized from a water/alcohol mixture, such as a mixture of water and isopropanol. In embodiments, the may further include a base, such as an inorganic base, such that the pH is less than about 7.0 to prevent decomposition of the deoxyArbutin compound. In embodiments, the recrystallization process may take place in the presence of an antioxidant. Suitable antioxidants are described in further detail below; one exemplary antioxidant is ascorbic acid. It should be noted that in the case of acidic antioxidants such as ascorbic acid, care should be taken to adjust amounts of the antioxidant and the base to maintain a pH of less than about 7.0. The recrystallization process may take place at low temperature, such as a temperature of less than bout 20° C., less than about 10° C. or about 5° C. Once the recrystallized product is obtained, it may be dried at a temperature of less than about 32° C. (e.g., about room temperature) to avoid heat-induced degradation of the deoxyArbutin compound.

Amounts of deoxyArbutin Compounds

A deoxyArbutin compound (e.g., a compound of formula I, II or III) may be included in a composition in amounts of about 0.5 wt. % to about 10 wt. %, or about 1.0 wt. % to about 5.0 wt. %. In embodiments, a single deoxyArbutin compound is included in a composition in an amount of about 0.5 wt. % to about 10 wt. %, or about 1.0 wt. % to about 5.0 wt. %. In embodiments, a combination of one or more deoxyArbutin compounds may be included in a composition in a total amount of about 0.5 wt. % to about 10 wt. %, or from about 1 wt. % to about 5.0 wt. %.

For example, a deoxyArbutin compound or a combination of deoxyArbutin compounds may be included in a composition in amounts of up to about 0.5 wt. %, up to about 1.0 wt. %, up to about 1.5 wt. %, up to about 2.0 wt. %, up to about 2.5 wt. %, up to about 3.0 wt. %, up to about 3.5 wt. %, up to about 4.0 wt. %, up to about 5.5 wt. %, up to about 6.0 wt. %, up to about 6.5 wt. %, up to about 7.0 wt. %, up to about 7.5 wt. %, up to about 8.0 wt. %, up to about 8.5 wt. %, up to about 9.0 wt. %, up to about 9.5 wt. %, up to about 10 wt. %, at least about 0.5 wt. %, at least about 1.0 wt. %, at least about 1.5 wt. %, at least about 2.0 wt. %, at least about 2.5 wt. %, at least about 3.0 wt. %, at least about 3.5 wt. %, at least about 4.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10 wt. %, about 0.5 wt. %, about 1.0 wt. %, about 1.5 wt. %, about 2.0 wt. %, about 2.5 wt. %, about 3.0 wt. %, about 3.5 wt. %, about 4.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, or about 10 wt. %. In embodiments, a composition may include a deoxyArbutin compound at an amount that is safe and effective for skin lightening, while producing a stable composition that does not substantially change in color.

### Antioxidants/Radical Scavengers

Compositions of the present disclosure may include at least one antioxidant/radical scavenger. The inclusion of an antioxidant may increase the skin lightening benefits of the composition, and may protect the deoxyArbutin compound from oxidative damage. Inclusion of antioxidants may also prevent discoloration (e.g., browning) of a composition due to such damage.

Exemplary antioxidants include but are not limited to ascorbic acid (vitamin C) and salts and esters thereof (e.g., sodium ascorbate, ascorbyl phosphate and salts thereof such as magnesium ascorbyl phosphate, ascorbyl esters of fatty acids such as ascorbyl palmitate), tocopherol (vitamin E) and salts and esters thereof (e.g., tocopheryl acetate, tocopheryl phosphate), butylated hydroxy benzoic acids and their salts, butylated hydroxytoluene, butylated hydroxyanisole, 6-hydroxy-2,5,7,8-tetramethylchroman-2-carboxylic acid (commercially available under the tradename Trolox™), gallic acid and its alkyl esters (e.g., propyl gallate), uric acid and its salts and alkyl esters, sorbic acid and its salts, amines (e.g., N,N-diethylhydroxylamine, amino-guanidine), sulfhydryl compounds (e.g., glutathione), sodium metabisulfite, and dihydroxy fumaric acid and its salts may be used.

Combinations of antioxidants may also be included in the compositions described herein. In embodiments, a composition may include at least two antioxidants, at least three antioxidants, at least four antioxidants, at least five antioxidants or more. In some embodiments, a composition comprises two antioxidants. In some embodiments, a composition comprises three antioxidants. In some embodiments, a composition comprises four antioxidants. In some embodiments, a composition comprises five antioxidants. For example, in one embodiment, a composition may include at least one salt or ester of ascorbic acid and at least one salt or ester of tocopherol. For example, a composition may include tocopheryl acetate (e.g., dl-alpha-tocopheryl acetate), an ascorbyl phosphate compound (e.g., magnesium ascorbyl phosphate), and ascorbyl palmitate. A composition may further include additional antioxidants such as butylated hydroxytoluene and sodium metabisulfite.

One or more antioxidants may be added to the compositions. For example, an antioxidant may be included in a composition at an amount of about 0.01 wt. % to about 3.0 wt. %, or from about 0.05% to about 0.6 wt. %. In embodiments, each antioxidant is included at an amount of about 0.01 wt. % to about 3.0 wt. %, or from about 0.05% to about 0.6 wt. %. In embodiments, a combination of antioxidants is included at a total amount of about 0.01 wt. % to about 3.0 wt. %, or from about 0.05% to about 0.6 wt. %.

In embodiments, an antioxidant or a mixture of antioxidants may be included in a composition at an amount of up to about 0.01 wt. %, up to about 0.02 wt. %, up to about 0.03 wt. %, up to about 0.04 wt. %, up to about 0.05 wt. %, up to about 0.06 wt. %, up to about 0.07 wt. %, up to about 0.08 wt. %, up to about 0.09 wt. %, up to about 0.10 wt. %, up to about 0.11 wt. %, up to about 0.12 wt. %, up to about 0.13 wt. %, up to about 0.14 wt. %, up to about 0.15 wt. %, up to about 0.16 wt. %, up to about 0.17 wt. %, up to about 0.18 wt. %, up to about 0.19 wt. %, up to about 0.20 wt. %, up to about 0.25 wt. %, up to about 0.30 wt. %, up to about 0.35 wt. %, up to about 0.40 wt. %, up to about 0.45 wt. %, up to about 0.50 wt. %, up to about 0.55 wt. %, up to about 0.60 wt. %, at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.10 wt. %, at least about 0.11 wt. %, at least about 0.12 wt. %, at least about 0.13 wt. %, at least about 0.14 wt. %, at least about 0.15 wt. %, at least about 0.16 wt. %, at least about 0.17 wt. %, at least about 0.18 wt. %, at least about 0.19 wt. %, at least about 0.20 wt. %, at least about 0.25 wt. %, at least about 0.30 wt. %, at least about 0.35 wt. %, at least about 0.40 wt. %, at least about 0.45 wt. %, at least about 0.50 wt. %, . %, at least about 0.55 wt. %, at least about 0.60 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.10 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.20 wt. %, about 0.25 wt. %, about 0.30 wt. %, about 0.35 wt. %, about 0.40 wt. %, about 0.45 wt. %, about 0.50 wt. %, about 0.55 wt. %, or about 0.60 wt. %.

### Glycols

Compositions of the present disclosure may include at least one glycol, which may act as a solvent and/or a viscosity decreasing agent. As used herein, a “glycol” refers to a compound having vicinal hydroxyl groups (two hydroxyl groups on adjacent carbon atoms). In embodiments the glycol may further include additional hydroxyl groups, such as in the case of glycerol. In embodiments, the glycol may be a glycol ether such as, for example, ethylene glycol monomethyl ether. Examples of suitable glycols include but are not limited to ethylene glycol, propylene glycols (e.g., 1,2-propylene glycol and 1,3-propylene glycol), butylene glycols (e.g., 1,3-butylene glycol), polyethylene glycols, polypropylene glycols, butylene glycols, ethylene glycol ethers, propylene glycol ethers, glycerol, 1,2,4-butanetriol, and mixtures thereof. Suitable glycol ethers include but are not limited to ethylene glycol monomethyl ether (2-methoxyethanol), ethylene glycol monoethyl ether (2-ethoxyethanol), ethylene glycol monopropyl ether (2-propoxyethanol), ethylene glycol monoisopropyl ether (2-isopropoxyethanol), ethylene glycol monobutyl ether (2-butoxyethanol), ethylene glycol monophenyl ether (2-phenoxyethanol), ethylene glycol monobenzyl ether (2-benzyloxyethanol), diethylene glycol monomethyl ether (2-(2-methoxyethoxy)ethanol, methyl carbitol), diethylene glycol monoethyl ether (2-(2-ethoxyethoxy)ethanol, ethoxydiglycol), diethylene glycol mono-n-butyl ether (2-(2-butoxyethoxy)ethanol), ethylene glycol dimethyl ether (dimethoxyethane), ethylene glycol diethyl ether (diethoxyethane), and ethylene glycol dibutyl ether (dibutoxyethane). A suitable glycol may be diethylene glycol monoethyl ether (2-(2-ethoxyethoxy)ethanol, ethoxydiglycol). A suitable combination of glycols may be diethylene glycol monoethyl ether and 1,3-butylene glycol.

One or more glycols may be added to the compositions. For example, a glycol may be included in a composition at an amount of about 0.1 wt. % to about 10 wt. %, about 0.5 wt. % to about 7.5 wt. %, or from about 0.5 wt. % to about 5.0 wt. %. In embodiments, each glycol is included at an amount of about 0.1 wt. % to about 10 wt. %, about 0.5 wt. % to about 7.5 wt. %, or from about 0.5 wt. % to about 5.0 wt. %. In embodiments, a combination of glycols is included at a total amount of about 0.1 wt. % to about 10 wt. %, about 0.5 wt. % to about 7.5 wt. %, or from about 0.5 wt. % to about 5.0 wt. %.

In embodiments, a glycol or a mixture of glycols may be included in a composition at an amount of at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.5 wt. %, at least about 2.0 wt. %, at least about 2.5 wt. %, at least about 3.0 wt. %, at least about 3.5 wt. %, at least about 4.0 wt. %, at least about 4.5 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10 wt. %, up to about 0.1 wt. %, up to about 0.2 wt. %, up to about 0.3 wt. %, up to about 0.4 wt. %, up to about 0.5 wt. %, up to about 0.6 wt. %, up to about 0.7 wt. %, up to about 0.8 wt. %, up to about 0.9 wt. %, up to about 1.0 wt. %, up to about 1.5 wt. %, up to about 2.0 wt. %, up to about 2.5 wt. %, up to about 3.0 wt. %, up to about 3.5 wt. %, up to about 4.0 wt. %, up to about 4.5 wt. %, up to about 5.0 wt. %, up to about 5.5 wt. %, up to about 6.0 wt. %, up to about 6.5 wt. %, up to about 7.0 wt. %, up to about 7.5 wt. %, up to about 8.0 wt. %, up to about 8.5 wt. %, up to about 9.0 wt. %, up to about 9.5 wt. %, up to about 10 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, or about 10 wt. %.

### Chelators

In embodiments, a chelating agent is included in the composition. Chelating agents are capable of removing a metal ion from a system by forming a complex, so that the metal ion cannot readily participate in or catalyze chemical reactions. The inclusion of a chelating agent may increase the skin lightening benefits and/or the stability of the composition.

Chelating agents are known in the art and a non-exhaustive list thereof can be found in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996). Examples of chelating agents include, but are not limited to, phosphonic acid and phosphonates, phosphates, aminocarboxylates and their derivatives, pyrophosphates, ethylenediamine and ethylenetriamine derivatives, hydroxyacids, and mono-, di-, and tri-carboxylates and their corresponding acids. Other chelating agents include nitroloacetates and their derivatives. Examples of aminocarboxylates include amino acetates and salts thereof. Suitable amino acetates include N-hydroxyethylaminodiacetic acid, hydroxyethylenediaminetetraacetic acid, nitrilotriacetic acid (NTA), ethylenediaminetetraacetic acid (EDTA), N-hydroxyethyl-ethylenediaminetriacetic acid (HEDTA), tetrasodium ethylenediaminetetraacetic acid (EDTA), diethylenetriaminepentaacetic acid (DTPA), alanine-N,N-diacetic acid, n-hydroxyethyliminodiacetic acid, and the like, as well as salts thereof (e.g., ammonium salts, alkali metal salts and alkaline earth metal salts), and mixtures thereof. Suitable aminophosphates include nitrilotrismethylene phosphates and other aminophosphates with alkyl or alkaline groups with less than 8 carbon atoms. Exemplary polycarboxylates include iminodisuccinic acids (IDSs), sodium polyacrylates, citric acid, gluconic acid, oxalic acid, salts thereof, mixtures thereof, and the like. Additional polycarboxylates include citric or citrate-type chelating agents, polymeric polycarboxylate, and acrylic or polyacrylic acid-type chelating agents. Additional chelating agents include polyaspartic acid or co-condensates of aspartic acid with other amino acids, C4-C25-mono- or -dicarboxylic acids and C4-C25-mono- or -diamines. Exemplary polymeric polycarboxylates include polyacrylic acid, maleic/olefin copolymer, acrylic/maleic copolymer, polymethacrylic acid, acrylic acid-methacrylic acid copolymers, hydrolyzed polyacrylamide, hydrolyzed polymethacrylamide, hydrolyzed polyamide-methacrylamide copolymers, hydrolyzed polyacrylonitrile, hydrolyzed polymethacrylonitrile, hydrolyzed acrylonitrile-methacrylonitrile copolymers, and the like. A suitable chelating agent is EDTA.

One or more chelating agents may be added to the compositions. For example, a chelating agent may be included in a composition at an amount of about 0.01 to about 5.0 wt. %, about 0.025 to about 3.0 wt. %, or about 0.05 wt. % to about 1.0 wt. %. In embodiments, each chelating agent is included at an about 0.01 to about 5.0 wt. %, about 0.025 to about 3.0 wt. %, or about 0.05 wt. % to about 1.0 wt. %. In embodiments, a combination of chelating agents is included at a total amount of about 0.01 to about 5 wt. %, about 0.025 to about 3.0 wt. %, or about 0.05 wt. % to about 1.0 wt. %.

In embodiments, a chelating agent or a mixture of chelating agents may be included in a composition at an amount of up to about 0.01 wt. %, up to about 0.02 wt. %, up to about 0.03 wt. %, up to about 0.04 wt. %, up to about 0.05 wt. %, up to about 0.06 wt. %, up to about 0.07 wt. %, up to about 0.08 wt. %, up to about 0.09 wt. %, up to about 0.10 wt. %, up to about 0.15 wt. %, up to about 0.20 wt. %, up to about 0.25 wt. %, up to about 0.30 wt. %, up to about 0.35 wt. %, up to about 0.40 wt. %, up to about 0.45 wt. %, up to about 0.50 wt. %, up to about 0.55 wt. %, up to about 0.60 wt. %, up to about 0.70 wt. %, up to about 0.80 wt. %, up to about 0.90 wt. %, up to about 1.0 wt. %, at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.10 wt. %, at least about 0.15 wt. %, at least about 0.20 wt. %, at least about 0.25 wt. %, at least about 0.30 wt. %, at least about 0.35 wt. %, at least about 0.40 wt. %, at least about 0.45 wt. %, at least about 0.50 wt. %, at least about 0.55 wt. %, at least about 0.60 wt. %, at least about 0.70 wt. %, at least about 0.80 wt. %, at least about 0.90 wt. %, at least about 1.0 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.10 wt. %, about 0.15 wt. %, about 0.20 wt. %, about 0.25 wt. %, about 0.30 wt. %, about 0.35 wt. %, about 0.40 wt. %, about 0.45 wt. %, about 0.50 wt. %, about 0.55 wt. %, about 0.60 wt. %, about 0.70 wt. %, about 0.80 wt. %, about 0.90 wt. %, or about 1.0 wt. %.

### Emulsifiers/Surfactants

Compositions may include emulsifiers, such as surfactants. Emulsifiers may be nonionic, anionic, cationic, zwitterionic or amphoteric. Suitable emulsifiers are disclosed in, for example, U McCutcheon's Detergents and Emulsifiers, North American Edition, pages 317-324 (1986), incorporated herein by reference.

Suitable non-ionic surfactants include, for example, mono- and di-alkanolamines such as, for example, cocamide monoethanolamine and cocamide diethanolamine, amine oxides, alkyl polyglucosides, ethoxylated silicones, ethoxylated alcohols, ethoxylated carboxylic acids, ethoxylated fatty acids, ethoxylated amines, ethoxylated amides, ethoxylated alkylolamides, ethoxylated alkylphenols, ethoxylated glyceryl esters, ethoxylated sorbitan esters, ethoxylated phosphate esters, glycol stearate, glyceryl stearate, and combinations thereof. For example, suitable non-ionic surfactants include ethoxylated alcohols, which include polyethylene glycol ethers of alcohols such as fatty alcohols. For example, a class of non-ionic surfactants commonly known as steareths are polyethylene glycol ethers of stearic acid; examples include steareth-2, steareth-10, steareth-20, steareth-20, and steareth-21. Such compounds are widely commercially available and include, for example, Brij™ non-ionic polyoxyethlene surfactants (available from, e.g., Croda).

Suitable anionic surfactants include, for example, alkyl sulfates, alkyl ether sulfates, alkyl aryl sulfonates (e.g., a linear alkyl benzene sulfonate), alpha-olefin sulfonates, alkali metal or ammonium salts of alkyl sulfates, alkali metal or ammonium salts of alkyl ether sulfates, alkyl phosphates, silicone phosphates, alkyl glyceryl sulfonates, alkyl sulfosuccinates, alkyl taurates, acyl taurates, alkyl sarcosinates, acyl sarcosinates, sulfoacetates, alkyl phosphate esters, mono alkyl succinates, monoalkyl maleates, sulfoacetates, acyl isethionates, alkyl carboxylates, phosphate esters, sulfosuccinates (e.g., sodium dioctylsulfosuccinate), and combinations thereof. Some non-limiting examples of anionic surfactants include sodium lauryl sulfate, sodium lauryl ether sulfate, ammonium lauryl sulfosuccinate, ammonium lauryl sulfate, ammonium lauryl ether sulfate, sodium dodecylbenzene sulfonate, triethanolamine dodecylbenzene sulfonate, sodium cocoyl isethionate, sodium lauroyl isethionate, sodium N-lauryl sarcosinate, and combinations thereof.

Suitable cationic surfactants include, for example, alkyl ammonium salts, polymeric ammonium salts, alkyl pyridinium salts, aryl ammonium salts, alkyl aryl ammonium salts, silicone quaternary ammonium compounds, and combinations thereof. Some non-limiting examples of cationic surfactants include behenyl trimonium chloride, stearalkonium chloride, distearalkonium chloride, chlorohexidine digluconate, polyhexamethylene biguanide (PHMB), cetyl pyridinium chloride, benzammonium chloride, benzalkonium chloride, and combinations thereof.

Suitable amphoteric surfactants include, for example, betaines, alkylamido betaines, sulfobetaines, N-alkyl betaines, sultaines, amphoacetates, amophodiacetates, imidazoline carboxylates, sarcosinates, acylamphoglycinates, such as cocamphocarboxyglycinates and acylamphopropionates, and combinations thereof. Some non-limiting examples of amphoteric surfactants include cocamidopropyl betaine, lauramidopropyl betaine, meadowfoamamidopropyl betaine, sodium cocoyl sarcosinate, sodium cocamphoacetate, disodium cocoamphodiacetate, ammonium cocoyl sarcosinate, sodium cocoamphopropionate, and combinations thereof.

Suitable zwitterionic surfactants include, for example, alkyl amine oxides, silicone amine oxides, and combinations thereof. Some non-limiting examples of suitable zwitterionic surfactants include, for example, 4-[N,N-di(2-hydroxyethyl)-N-octadecylammonio]-butane-1-carboxylate, S—[S-3-hydroxypropyl-5-hexadecylsulfonio]-3-hydroxypentane-1-sulfate, 3-[P,P-diethyl-P-3,6,9-trioxatetradexopcylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)propane-1-sulfonate, 3-(N,N-dimethyl-N-hexadecylammonio)-2-hydroxypropane-1-sulfonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulfonio]-propane-1-phosphate-, 3-[P,P-dimethyl-P-dodecylphosphonio]-propane-1-phosphonate, 5-[N,N-di(3-hydroxypropyl)-N-hexadecylammonio]-2-hydroxy-pentane-1-sulfate, and combinations thereof.

Additional emulsifiers include fatty alcohols such as, for example, fatty alcohols having from about 8 to about 20 carbon atoms. Suitable fatty alcohols include but are not limited to capryl alcohol, 2-ethyl hexanol, pelargonic alcohol, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, isostearyl alcohol, elaidyl alcohol, oleyl alcohol, linoleyl alcohol, elaidolinoleyl alcohol, linolenyl alcohol, elaidolinolenyl alcohol, ricinoleyl alcohol, nonadecyl alcohol, arachidyl alcohol and behenyl alcohol.

Other suitable emulsifiers include copolymers, such as acrylate copolymers. For example, suitable emulsifiers include acrylates/alkyl acrylates crosspolymers, such as an acrylates/C₁₀-C₃₀ alkyl acrylate crosspolymer. Such polymers are sold under the trade name Pemulen™ TR-1, e.g., from Lubrizol.

One or more emulsifiers may be added to the compositions. For example, an emulsifier may be included at an amount of about 0.01 to about 10 wt. %, about 0.05 to about 7.5 wt. %, or about 0.1 wt. % to about 5.0 wt. %. In embodiments, each emulsifier is included at an about 0.01 to about 10 wt. %, about 0.05 to about 7.5 wt. %, or about 0.1 wt. % to about 5.0 wt. %. In embodiments, a combination of emulsifiers is included at a total amount of about 0 about 0.01 to about 10 wt. %, about 0.05 to about 7.5 wt. %, or about 0.1 wt. % to about 5.0 wt. %.

In embodiments, an emulsifier or mixture of emulsifiers may be included in a composition at an amount of at least about 0.1 wt. %, at least about 0.2 wt. %, at least about 0.3 wt. %, at least about 0.4 wt. %, at least about 0.5 wt. %, at least about 0.6 wt. %, at least about 0.7 wt. %, at least about 0.8 wt. %, at least about 0.9 wt. %, at least about 1.0 wt. %, at least about 1.5 wt. %, at least about 2.0 wt. %, at least about 2.5 wt. %, at least about 3.0 wt. %, at least about 3.5 wt. %, at least about 4.0 wt. %, at least about 4.5 wt. %, at least about 5.0 wt. %, at least about 5.5 wt. %, at least about 6.0 wt. %, at least about 6.5 wt. %, at least about 7.0 wt. %, at least about 7.5 wt. %, at least about 8.0 wt. %, at least about 8.5 wt. %, at least about 9.0 wt. %, at least about 9.5 wt. %, at least about 10 wt. %, up to about 0.1 wt. %, up to about 0.2 wt. %, up to about 0.3 wt. %, up to about 0.4 wt. %, up to about 0.5 wt. %, up to about 0.6 wt. %, up to about 0.7 wt. %, up to about 0.8 wt. %, up to about 0.9 wt. %, up to about 1.0 wt. %, up to about 1.5 wt. %, up to about 2.0 wt. %, up to about 2.5 wt. %, up to about 3.0 wt. %, up to about 3.5 wt. %, up to about 4.0 wt. %, up to about 4.5 wt. %, up to about 5.0 wt. %, up to about 5.5 wt. %, up to about 6.0 wt. %, up to about 6.5 wt. %, up to about 7.0 wt. %, up to about 7.5 wt. %, up to about 8.0 wt. %, up to about 8.5 wt. %, up to about 9.0 wt. %, up to about 9.5 wt. %, up to about 10 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, or about 10 wt. %.

### Conditioning Agents

The compositions may include at least one conditioning agent such as an emollient, humectant, occlusive agent, or other moisturizer to provide moisturizing, skin softening, skin barrier maintenance, anti-irritation, or other skin health benefits. Some non-limiting examples of emollients include stearoxytrimethylsilane, alkyl benzoate, silicone oils, dimethicone, myristyl myristate, cetyl myristate, glyceryl dioleate, methyl laurate, PPG-9 laurate, octyl palmitate, lanolin, propylene glycol, glycerol, fatty acids, natural oils such as sunflower, almond, mineral, canola, sesame, soybean, wheat germ, corn, peanut and olive, isopropyl myristate, myristyl alcohol, aloe vera, hydrolyzed silk protein, Vitamin E, stearyl alcohol, isopropyl palmitate, sorbitol, amino acid complexes, and polyethylene glycol. Some non-limiting examples of humectants include hydroxyethyl urea, agarose, arginine PCA, ethylhexylglycerin, fructose, glucose, glutamic acid, glycerol, honey, lactose, maltose, propylene glycol (e.g., 1,2-propylene glycol), butylene glycol (e.g., 1,3-butylene glycol), polyethylene glycols and ethers thereof, polypropylene glycols and ethers thereof (e.g., polypropylene glycol ethers such as polypropylene glycol-14 butyl ether), sorbitol and mixtures thereof. Some non-limiting examples of occlusive agents include petrolatum, shea butter, alkyl dimethicones, avocado oil, balm mint oil, canola oil, cod liver oil, corn oil, methicone, mineral oil, olive oil, phenyl trimethicone, trimyristin, soybean oil, glycol distearate, stearyl stearate, synthetic wax, or mixtures thereof. Some non-limiting examples of other moisturizers include cholesterol, cystine, hyaluronic acid, keratin, lecithin, egg yolk, glycine, PPG-12, panthenol, retinol, vegetable oil, and mixtures thereof. Some non-limiting examples of anti-irritants include bisabolol and panthenol.

One or more conditioning agents may be added to the compositions. For example, a conditioning agent may be included in a composition at an amount of about 0.01 to about 10 wt. %, about 0.05 to about 7.5 wt. %, or about 0.1 wt. % to about 5.0 wt. %. In embodiments, each conditioning agent is included in an amount of about 0.01 to about 10 wt. %, about 0.05 to about 7.5 wt. %, or about 0.1 wt. % to about 5.0 wt. %. In embodiments, a combination of conditioning agents is included at a total amount of 0.01 to about 10 wt. %, about 0.05 to about 7.5 wt. %, or about 0.1 wt. % to about 5.0 wt. %.

In embodiments, a conditioning agent or mixture of conditioning agents may be included in a composition at an amount of at least about 0.5 wt %, at least about 0.75 wt %, at least about 1.0 wt %, at least about 1.5 wt %, at least about 2.0 wt %, at least about 2.5 wt %, at least about 3.0 wt %, at least about 3.5 wt %, at least about 4.0 wt %, at least about 4.5 wt %, at least about 5.0 wt %, up to about 0.5 wt %, up to about 0.75 wt %, up to about 1.0 wt %, up to about 1.5 wt %, up to about 2.0 wt %, up to about 2.5 wt %, up to about 3.0 wt %, up to about 3.5 wt %, up to about 4.0 wt %, up to about 4.5 wt %, up to about 5.0 wt %, about 0.5 wt %, about 0.75 wt %, about 1.0 wt %, about 1.5 wt %, about 2.0 wt %, about 2.5 wt %, about 3.0 wt %, about 3.5 wt %, about 4.0 wt %, about 4.5 wt %, or about 5.0 wt %.

### Preservatives

The compositions may include at least one preservative, which may have anti-microbial activity. Suitable preservatives may be efficacious against a broad spectrum of microbes. Examples of preservatives include, but not limited to, benzalkonium chloride, benzoic acid, benzoxonium chloride, benzyl alcohol, 2-bromo-2-nitropropane-1,3-diol, 5-bromo-5-nitro-1,3-dioxane, bromochlorophene, camphor benzalkonium methosulfate, captan, cetrimonium bromide, cetrimonium chloride, cetylpyridinium chloride, climbazol, chloracetamide, chlorhexidine and its salts, p-chloro-m-cresol, chlorphenesin, chloroxylenol, chlorophen, chlorobutanol, o-cymen-5-ol, dehydroacetic acid, dibromodicyanobutan, dibromohexamidin, dibromopropamidin, dichlorobenzyl alcohol, dichlorophenyl imidazoldioxolan, dimethyloxazolidin, DMDM hydantoin, dodecylguanidine acetate, hexamidine diisothionate, hexachlorophen, hexetidin, iodopropynyl butylcarbamate, lauryl isoquinolinium bromide, methyldibromo glutaronitrile, methylolchloracetamide, parabens such as methylparaben, ethylparaben, propylparaben and butylparaben, phenethyl alcohol, phenoxyethanol, phenoxypropanol, o-phenylphenol, piroctone olamine, polyaminopropyl biguanide, potassium sorbate, potassium undecylenoyl hydrolyzed collagen, quaternium-15, salicylic acid, sodium benzoate, sodium dehydroacetate, sodium hydroxymethylglycinate, sodium o-phenylphenate, sorbic acid, triclocarban, triclosan, undecylenic acid and its derivatives, zinc cysteate, zinc gluconate, zinc pyrithione, and zinc sulfate. Derivatives of undecylenic acid useful as anti-microbial agents are e.g. esters, such as methyl ester, isopropyl ester, glyceryl ester, ethoxylated soya sterol ester, or ethoxylated PHB ester, or amides, such as monoethanolamide, monoethanolamide derivatives such as monoethanolamide (MEA) sulfosuccinate salts, diethanolamide, protein condensates, e.g. potassium undecylenoyl hydrolyzed animal collagen, and quaternized 3-aminopropyl-amide, e.g. undecylenamidopropyltrimonium methosulfate. Specific examples of suitable fungicidal/fungistatic agents include, without limitation, dithiocarbamates, phthalimides, dicarboximides, organophosphates, benzimidazoles, carboxanilides, phenylamides, phosphites, and the like.

One or more preservatives may be added to the compositions. For example, a preservative may be included in a composition at an amount of about 0.01 to about 5.0 wt. %, about 0.05 to about 2.5 wt. %, or about 0.1 wt. % to about 1.0 wt. %. In embodiments, each preservative is included in an amount of about 0.01 to about 5.0 wt. %, about 0.05 to about 2.5 wt. %, or about 0.1 wt. % to about 1.0 wt. %. In embodiments, a combination of preservatives is included at a total amount of about 0.01 to about 5.0 wt. %, about 0.05 to about 2.5 wt. %, or about 0.1 wt. % to about 1.0 wt. %.

In embodiments, a preservative or mixture of preservatives may be included in a composition at an amount of at least about of up to about 0.01 wt. %, up to about 0.02 wt. %, up to about 0.03 wt. %, up to about 0.04 wt. %, up to about 0.05 wt. %, up to about 0.06 wt. %, up to about 0.07 wt. %, up to about 0.08 wt. %, up to about 0.09 wt. %, up to about 0.10 wt. %, up to about 0.15 wt. %, up to about 0.20 wt. %, up to about 0.25 wt. %, up to about 0.30 wt. %, up to about 0.35 wt. %, up to about 0.40 wt. %, up to about 0.45 wt. %, up to about 0.50 wt. %, up to about 0.55 wt. %, up to about 0.60 wt. %, up to about 0.65 wt. %, up to about 0.70 wt. %, up to about 0.75 wt. %, up to about 0.80 wt. %, up to about 0.85 wt. %, up to about 0.90 wt. %, up to about 0.95 wt. %, up to about 1.0 wt. %, at least about 0.01 wt. %, at least about 0.02 wt. %, at least about 0.03 wt. %, at least about 0.04 wt. %, at least about 0.05 wt. %, at least about 0.06 wt. %, at least about 0.07 wt. %, at least about 0.08 wt. %, at least about 0.09 wt. %, at least about 0.10 wt. %, at least about 0.15 wt. %, at least about 0.20 wt. %, at least about 0.25 wt. %, at least about 0.30 wt. %, at least about 0.35 wt. %, at least about 0.40 wt. %, at least about 0.45 wt. %, at least about 0.50 wt. %, at least about 0.55 wt. %, at least about 0.60 wt. %, at least about 0.65 wt. %, at least about 0.70 wt. %, at least about 0.75 wt. %, at least about 0.80 wt. %, at least about 0.85 wt. %, at least about 0.90 wt. %, at least about 0.95 wt. %, at least about 1.0 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.10 wt. %, about 0.15 wt. %, about 0.20 wt. %, about 0.25 wt. %, about 0.30 wt. %, about 0.35 wt. %, about 0.40 wt. %, about 0.45 wt. %, about 0.50 wt. %, about 0.55 wt. %, about 0.60 wt. %, about 0.65 wt. %, about 0.70 wt. %, about 0.75 wt. %, about 0.80 wt. %, about 0.85 wt. %, about 0.90 wt. %, about 0.95 wt. %, or about 1.0 wt. %.

### Sunscreens and Sunblocks

Regulation of skin darkening resulting from exposure to ultraviolet light can be achieved by using including sunscreening agents or sunblocks in the compositions. Useful sunblocks include, for example, zinc oxide and titanium dioxide.

A wide variety of conventional sunscreening agents are suitable for use in compositions. Sagarin et al. at Chapter VIII, pages 189 et seq., of Cosmetics Science and Technology (1972), disclose numerous suitable agents. Specific suitable sunscreening agents include, for example: p-aminobenzoic acid ands its salts and derivatives (ethyl, isobutyl, glyceryl esters; p-dimethylaminobenzoic acid); anthranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters); cinnamic acid derivatives (menthyl and benzyl esters, α-phenyl cinnamonitrile; butyl cinnamoyl pyruvate); dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives (e.g., hexaethylether); (butyl carbotol) (6-propyl piperonyl)ether; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; 4,4′-t-butylmethoxydibenzoylmethane; and etocrylene.

Suitable sunscreens include 2-ethylhexyl-p-methoxycinnamate, 4,4′-t-butylmethoxydibenzoylmethane, 4,4′-t-butylmethoxydibenzoylmethane, 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl-p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid and mixtures thereof.

Also useful in the compositions are sunscreens such as those disclosed in U.S. Pat. No. 4,937,370 and U.S. Pat. No. 4,999,186. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. Suitable members of this class of sunscreening agents include 4-N,N-(2-ethylhexyl)methylaminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl) methylaminobenzoic acid ester with 4-hydroxydibenzoylmethane; 4-N,N-(2-ethylhexyl)methylaminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof.

A safe and effective amount of sunscreen may be used in the compositions useful in this invention. The sunscreening agent must be compatible with the skin lightening agent. The composition may include from about 1 wt. % to about 20 wt. %, or from about 2 wt. % to about 10 wt. %, of a sunscreening agent. Exact amounts will vary depending upon the sunscreen chosen and the desired Sun Protection Factor (SPF).

An agent may also be added to any of the compositions useful in this invention to improve the skin substantivity of those compositions, particularly to enhance their resistance to being washed off by water, or rubbed off. A suitable agent which may provide this benefit is a copolymer of ethylene and acrylic acid. Compositions comprising this copolymer are disclosed in U.S. Pat. No. 4,663,157.

### Anti-Inflammatory Agents

In skin lightening compositions of the present disclosure, an anti-inflammatory agent may be included as an additional agent. The inclusion of an anti-inflammatory agent may, in some embodiments, enhance the skin lightening benefits of the compositions. The anti-inflammatory agent may protect in the UVA radiation range, and may provide some UVB protection as well. The topical use of anti-inflammatory agents reduces darkening of the skin resulting from chronic exposure to UV radiation. (See, e.g., U.S. Pat. No. 4,847,071 and U.S. Pat. No. 4,847,069.)

A safe and effective amount of an anti-inflammatory agent may be added to the compositions useful in this invention, e.g., from about 0.1 wt. % to about 10 wt. % or from about 0.5 wt. % to about 5 wt. %, of the composition. The exact amount of anti-inflammatory agent to be used in the compositions will depend on the particular anti-inflammatory agent utilized, as such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. A suitable steroidal anti-inflammatory for use is hydrocortisone.

A second class of anti-inflammatory agents which is useful in the compositions includes the nonsteroidal anti-inflammatory agents. The variety of compounds encompassed by this group are well-known to those skilled in the art. For detailed disclosure of the chemical structure, synthesis, side effects, etc., of non-steroidal anti-inflammatory agents, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, N.Y. (1974).

Specific non-steroidal anti-inflammatory agents useful in the composition invention include, but are not limited to:

1) oxicams, such as piroxicam, isoxicam, tenoxicam, sudoxicam, and CP-14,304;

2) salicylates, such as aspirin, disalcid, benorylate, trilisate, safapryn, solprin, diflunisal, and fendosal;

3) acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac;

4) fenamates, such as mefenamic, meclofenamic, flufenamic, niflumic, and tolfenamic acids;

5) propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and

6) pyrazoles, such as phenybutazone, oxyphenbutazone, feprazone, azapropazone, and trimethazone.

Mixtures of these non-steroidal anti-inflammatory agents may also be employed, as well as the pharmaceutically-acceptable salts and esters of these agents. For example, etofenamate, a flufenamic acid derivative, is particularly useful for topical application. Of the nonsteroidal anti-inflammatory agents, ibuprofen, naproxen, flufenamic acid, mefenamic acid, meclofenamic acid, piroxicam and felbinac are suitable.

Another class of anti-inflammatory agents which are useful in the compositions are the anti-inflammatory agents disclosed in U.S. Pat. No. 4,708,966. This patent discloses a class of nonsteroidal anti-inflammatory compounds which comprise specifically substituted phenyl compounds, especially substituted 2,6-di-tert-butyl phenol derivatives. For example, compounds selected from 4-(4′-pentyn-3′-one)-2,6-di-t-butylphenol; 4-(5′-hexynoyl)-2,6-di-t-butylphenol; 4-((S)-(−)-3′-methyl-5′-hexynoyl)-2,6-di-t-butylphenol; 4-((R)-(+)-3′-methyl-5′-hexynoyl)-2,6-di-1-butylphenol; and 4-(3′,3′-dimethoxypropionyl)-2,6-di-t-butylphenol may be suitable.

Yet another class of anti-inflammatory agents which are useful in the compositions are those disclosed in U.S. Pat. No. 4,912,248. This patent discloses compounds and diastereomeric mixtures of specific 2-naphthyl- containing ester compounds, especially naproxen ester and naproxol ester compounds, having two or more chiral centers. For example, compounds selected from (S)-naproxen-(S)-2-butyl ester, (S)-naproxen-(R)-2-butylester, (S)-naproxol-(R)-2-methyl butyrate, (S)-naproxol-(S)-2-methyl butyrate, diasteromeric mixtures of (S)-naproxen-(S)-2-butyl ester and (S)-naproxen-(R)-2-butyl ester, and diasteromeric mixtures of (S)-naproxol(R)-2-methyl butyrate and (S)-naproxol-(S)-2-methyl butyrate are useful in this invention.

Finally, so-called “natural” anti-inflammatory agents are useful in methods of this invention. For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), may be used.

### Retinoids

In embodiments, a retinoid, such as retinoic acid, is included as an active along with the skin lightening agent. The inclusion of a retinoid increases the skin lightening benefits of the composition. A suitable of a retinoid may be added to the compositions useful in this invention, e.g., from about 0.001 wt. % to about 2 wt. %, or from about 0.01 wt. % to about 1 wt. % of the composition. As used herein, “retinoid” includes all natural and/or synthetic analogs of Vitamin A or retinol-like compounds which possess the biological activity of Vitamin A in the skin as well as the geometric isomers and stereo isomers of these compounds, such as all-trans retinoic acid and 13-cis-retinoic acid.

### Other Components

Various other materials may also be present in the compositions. These include opacifiers (e.g., titanium dioxide), penetration enhancers, vitamins, fragrances, exfoliants, anti-acne agents, anti-aging agents.

For example, compositions may include a penetration enhancing agent. A suitable of penetration enhancing agent is from about 1% to about 5% of the composition. Examples of useful penetration enhancers, among others, are disclosed in U.S. Pat. Nos. 4,537,776, 4,552,872, 4,557,934, 4,130,667, 3,989,816, 4,017,641, and 4,954,487. Additional penetration enhancers are disclosed in Cooper, E. R., “Effect of Decylmethylsulfoxide on Skin Penetration”, Solution Behavior of Surfactants, Vol. 2 (Mittal and Fendler, eds.), Plenum Publishing Corp., 1982, pp. 1505-1516; Mahjour, M., B. Mauser, Z. Rashidbaigi & M. B. Fawzi, “Effect of Egg Yolk Lecithins and Commercial Soybean Lecithins on In Vitro Skin Permeation of Drugs”, Journal of Controlled Release, Vol. 14 (1990), pp. 243-252; Wong, O., J. Huntington, R. Konishi, J. H. Rytting & T. Higuchi, “Unsaturated Cyclic Ureas as New Nontoxic Biodegradable Transdermal Penetration Enhancers I: Synthesis”, Journal of Pharmaceutical Sciences, Vol. 77, No. 11 (November 1988), pp. 967-971; Williams, A. C. & B. W. Barry, “Terpenes and the Lipid-Protein-Partitioning Theory of Skin Penetration Enhancement”, Pharmaceutical Research Vol. 8, No. 1 (1991), pp. 17-24; and Wong, 0., J. Huntington, T. Nishihata & J. H. Rytting, “New Alkyl N,N-Dialkyl-Substituted Amino Acetates as Transdermal Penetration Enhancers”, Pharmaceutical Research, Vol. 6, No. 4 (1989), pp. 286-295. Other conventional skin care product additives may also be included in the compositions. For example, collagen, hyaluronic acid, elastin, hydrolysates, primrose oil, jojoba oil, epidermal growth factor, soybean saponins, mucopolysaccharides, and mixtures thereof may be used.

Various vitamins may also be included in the compositions useful in this invention. For example, Vitamin A and derivatives thereof, Vitamin B2, biotin, pantothenic, Vitamin D, and mixtures thereof may be used.

In some embodiments, a composition may include a pharmaceutically acceptable carrier, adjuvant, or vehicle. The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In embodiments, compositions may be substantially free of dyes and pigments.

### Solvents

Compositions of the present disclosure may include at least one acceptable solvent. Suitable solvents will be capable of having dispersed or dissolved therein the active compound(s) (e.g., a deoxyArbutin compound), additional formulation components, and will possess acceptable safety properties (e.g., irritation and sensitization characteristics). Water is a suitable solvent. Examples of suitable organic solvents include: polyvinylpyrrolidine, ethanol, isopropanol, butanediol, and mixtures thereof. Compositions may include about 50 wt. % to about 99 wt. %, or from about 70 wt. % to about 90 wt. % of an acceptable aqueous or organic solvent. The compositions may comprise less than about 99 wt. %, less than about wt. 95%, less than about 90 wt. %, less than about 85 wt. %, less than about 80 wt. %, or less than about 75 wt. % solvent. The compositions may comprise greater than about 70 wt. %, greater than about 75 wt. %, greater than about 80 wt. %, greater than about 85 wt. % solvent. The balance of the compositions may be solvent.

pH

The pH of compositions including deoxyArbutin compounds can affect both stability of the composition and efficacy. For example, lower pH may result in decomposition of the deoxyArbutin compound. In embodiments the pH may be about 6.0 to about 10.0, or about 7.0 to about 9.0. In embodiments, a composition may have a pH of at least about 6.0, at least about 6.5, at least about 7.0, at least about 7.5, at least about 8.0, at least about 8.5, or at least about 9.0.

Composition pH can be adjusted with acid or base, if necessary. Any acid or base compatible with the components of the composition can be used. Exemplary acids include citric acid, gluconic acid, lactic acid, acetic acid, and glycolic acid. Exemplary bases include sodium hydroxide, potassium hydroxide, and triethanolamine

### Dioxygen

Compositions may include only minimal amounts of dioxygen (O₂), as the presence of dioxygen may induce oxidative degradation of the deoxyArbutin compound, as well as potential discoloration (e.g., browning). Accordingly, compositions may be prepared under an inert atmosphere, such as an atmosphere of nitrogen or argon, as will be described in further detail below. The final compositions may include less than 100 ppm O₂, less than about 90 ppm O₂, less than about 80 ppm O₂, less than about 70 ppm O₂, less than about 60 ppm O₂, less than about 50 ppm O₂, less than about 45 ppm O₂, less than about 40 ppm O₂, less than about 35 ppm O₂, less than about 30 ppm O₂, less than about 25 ppm O₂, less than about 20 ppm O₂, less than about 15 ppm O₂, less than about 10 ppm O₂, less than about 5 ppm O₂, less than about 4 ppm O₂, less than about 3 ppm O₂, less than about 2 ppm O₂, or less than about 1 ppm O₂.

### Composition Stability

Compositions that include deoxyArbutin compounds are sensitive to potential color changes, such as browning, due to oxidative or thermal degradation of the deoxyArbutin compound (e.g., deoxyArbutin). The compositions described herein may be stable and minimize or eliminate degradation of deoxyArbutin compounds as well as discoloration.

Accordingly, compositions described herein may not substantially change in color when stored at ambient temperature and pressure for a period of time. The period of time may be at least about 1 day, at least about 2 days, at least about 3 days, at least about 4 days, at least about 5 days, at least about 6 days, at least about 7 days, at least about 2 weeks, at least about 3 weeks or at least about 4 weeks or more.

One method to quantify color changes includes measuring the CIELAB L* value over different periods of time (e.g. a period of time discussed above). CIELAB is one of several International Commission on Illumination (CIE) color spaces that defines the range of colors visible to the human eye, and should be readily known to those of skill in the art. The coordinate L* stands for lightness, while a* represents where the color is on the redness-greenness axis and b* stands for the color's position on the yellowness-blueness axis. For example, a color is defined as pure white when L*=100, a*=0 and b*=0. A color is defined as absolute black when L*=0, a*=0 and b*=0. The CIELAB values of compositions should remain relatively constant over long periods of time. For example, the compositions may show a change in L* of less than about 10.0, less than about 9.0, less than about 8.0, less than about 7.0, less than about 6.0, less than about 5.0, less than about 4.0, less than about 3.0, less than about 2.0 or less than about 1.0 over period of time, wherein the period of time may be at least about 1 day, at least about 2 days, at least about 3 days, at least about 4 days, at least about 5 days, at least about 6 days, at least about 7 days, at least about 2 weeks, at least about 3 weeks or at least about 4 weeks or more.

In embodiments, a composition may be substantially free of dyes or pigments that may mask discoloration.

Compositions described herein may also be substantially free of hydroquinone compounds when stored over time.

### Process for Preparing Compositions

In an aspect, the disclosure also provides a method of making a composition, comprising: mixing a compound of formula I as defined and described above and herein, an antioxidant, a glycol and a solvent under an inert atmosphere, and maintaining the pH at a range of about 6.0 to about 10.0.

To generate a composition that is stable and effective, care must be taken to avoid oxidation of the deoxyArbutin compound. Exposure to dioxygen, heat or acidic pH can result in decomposition of the deoxyArbutin compound, leading to discoloration of the product (e.g., browning). The components may be combined in a suitable order, and the pH carefully monitored the pH during the course of the entire process, to ensure that the pH stays in the range of about 6.0 to about 10.0, e.g., about 6.5 to about 8.5, or about 7.0 to about 8.0. pH can be adjusted through addition of a suitable acid or base, as addressed above. For example, during or following steps involving addition of an acidic compound, such as ascorbic acid, a base such as triethanolamine can be included to raise the pH.

It may also be beneficial to limit exposure to dioxygen throughout the process, to prevent oxidation of the deoxyArbutin compound. This can be accomplished using an inert gas such as nitrogen or argon during the mixing process.

The temperature during the entire process can be monitored to ensure stability. For example, during the step in which the deoxyArbutin compound is added, the temperature can be maintained at about 30° C. to about 80° C., e.g., from about 40° C. to about 70° C., about 50° C., about 55° C., about 60° C. or about 65° C. The temperature may be maintained at less than about 80° C., less than about 75° C., less than about 70° C., or less than about 65° C. The temperature may be maintained at greater than about 30° C., greater than about 35° C., greater than about 40° C., greater than about 45° C., or greater than about 50° C., greater than about 55° C., or greater than about 60° C.

A complete process may be as follows. A reaction vessel may be filled with an inert gas such as nitrogen or argon, followed by addition of water, as well as optional water-soluble components such as chelating agents, preservatives, solvents, conditioning agents and emulsifiers. The aqueous mixture may then be heated. In a separate vessel, an oil phase may be prepared that includes, for example, emulsifiers (e.g., fatty alcohols), and optional oil-soluble antioxidants, preservatives, solvents, and conditioning agents. The mixture may be heated if necessary to ensure formation of a homogeneous mixture. The oil phase may then be added to the aqueous phase with stirring, and the pH may be adjusted to a range of about 6.0 to about 10.0 (e.g., about 6.5 to about 8.5). The mixture may be optionally cooled, followed by addition of a pre-mixed solution of a deoxyArbutin compound in a solvent, such as a glycol solvent, wherein the pre-mixing takes place under an inert atmosphere. Following mixing until uniform, the remaining components may be added in one or more phases, taking care to ensure that the pH stays in the range of about 6.0 to about 10.0. A final step may be pH adjustment.

### Methods for Lightening Mammalian Skin

The present disclosure also encompasses methods for lightening mammalian skin. Such methods comprise the administration of a therapeutically effective amount of a composition to the skin or regions of the skin to be lightened. The amount of active agent and frequency of application will vary widely depending upon the pigmentation already in existence, the rate of further darkening of the skin or region of the skin, and the level of lightening desired. Additionally, when the product is used to treat hyperpigmented spots, it is expected that the application and amount will differ from the amount for lightening of general skin tone.

Any dose that is safe and effective may be used, and accordingly it is contemplated that for certain dosage forms, particularly topical dosage forms, the “dose” is any amount that provides the desired effect.

A therapeutically effective amount of skin lightening agent in a topical composition is applied, generally from about 1 μg to about 1000 mg per cm² skin per application, from about 2 μg to about 800 μg/cm² skin per application, from about 30 μg to about 700 μg/cm² skin, or from about 75 μg to about 250 μg/cm² skin. Application may from about four times a day to about twice a week, from about three times a day to about once every other day, from about once daily to about three times daily, once daily, twice daily or three times daily. Application for at least several days may be required to see a skin lightening effect (e.g., for at least 5 days, at least 6 days, at least 7 days, at least two weeks, at least three weeks or at least four weeks). After lightening is achieved, it may be possible to reduce the frequency and dosage frequency and dosage to a maintenance level, as desired. Such maintenance varies according to the individual, but may be from about 1/10 to about ½, or from about ⅕ to about ⅓ of the original dosage and/or frequency, as needed.

A suitable mode of administration is topical administration.

### Methods for Lightening Hair

The present disclosure also encompasses methods for lightening hair. Such methods comprise the administration of a therapeutically effective amount of a composition to the hair of a mammalian subject. The amount of active agent and frequency of application will vary widely depending upon the pigmentation already in existence, and the level of lightening desired.

Any dose that is safe and effective may be used, and accordingly it is contemplated that for certain dosage forms, particularly topical dosage forms, the “dose” is any amount that provides the desired effect.

### EXAMPLES

### Example 1

### Exemplary Compositions

Exemplary compositions are illustrated in Table 1, with amounts in wt. %.

TABLE 1
Exemplary compositions

| | Composition | | | | |
|---|---|---|---|---|---|
| | 1 | 2 | 3 | 4 | 5 |
| Purified water | 65.68 | 70.7 | 72.68 | 77.33 | 77.25 |
| Acrylates/C₁₀-C₃₀ alkyl | 0.2 | 0.2 | 0.2 | — | — |
| acrylate crosspolymer | | | | | |
| Disodium EDTA | 0.05 | 0.05 | 0.05 | 0.05 | 0.05 |
| Chlorphenesin | 0.3 | 0.3 | 0.3 | | 0.3 |
| 1,3-Butylene glycol | 1.5 | 1.5 | 1.5 | 1.5 | 1.5 |
| dl-alpha-tocopheryl acetate | 0.05 | 0.05 | 0.05 | — | — |
| Polypropylene glycol-14 butyl | 7.5 | 7.5 | 7.5 | 7.5 | 7.5 |
| ether | | | | | |
| Cetyl alcohol | 3 | 3 | 3 | 3 | 3 |
| Stearyl alcohol | 1.5 | 1.5 | 1.5 | 1.5 | 1.5 |
| Butylated hydroxytoluene | 0.1 | 0.1 | 0.1 | 2 | 2 |
| Steareth-21 | 2 | 2 | 2 | 1 | 1 |
| Steareth-2 | 1 | 1 | 1 | — | — |
| Triethanolamine | 1.76 | 1.76 | 1.76 | 1.77 | 0.75 |
| Purified water | 1 | 1 | 1 | — | — |
| Ethoxydiglycol | 6 | 4 | 4 | — | — |
| DeoxyArbutin | 6 | 3 | 1 | 3 | 3 |
| Sodium metabisulfite | 0.15 | 0.15 | 0.15 | 0.05 | 0.05 |
| Magnesium ascorbyl phosphate | 0.1 | 0.1 | 0.1 | 0.1 | 0.1 |
| Ascorbyl palmitate | 0.1 | 0.1 | 0.1 | 0.1 | 0.1 |
| Polyaminopropyl biguanide | 0.5 | 0.5 | 0.5 | — | — |
| (20%) | | | | | |
| Dimethicone | 1 | 1 | 1 | — | — |
| Benzyl alcohol | 0.5 | 0.5 | 0.5 | 0.5 | 0.5 |
| Lactic Acid | 0.01 | 0.01 | 0.01 | — | — |
| Methylparaben | — | — | — | 0.25 | — |
| Propylparaben | — | — | — | 0.1 | — |
| Sodium Hydroxide | — | — | — | 0.25 | — |
| Carbomer | — | — | — | — | 0.4 |
| Phenoxyethanol, | — | — | — | — | 1 |
| Ethylhexylglycerin | | | | | |

Compositions in Table 1 were prepared by first filling a vessel with an inert gas (e.g., nitrogen). Water was added and water-soluble components were added sequentially and the mixture was stirred until uniform. An oil phase was prepared separately and was added to the vessel with stirring to generate a uniform mixture. The pH was adjusted to 6.0-10.0. A mixture of deoxyArbutin and a suitable solvent (e.g., a glycol) were combined under an inert atmosphere in a separate vessel and mixed until uniform, and this mixture was then added to the main reaction vessel followed by mixing until uniform. After cooling, the remaining components were added in one or more phases, maintaining the pH at 6.0-10.0. The final step was pH adjustment to a final pH of 7.5-8.0.

### Example 2

An exemplary composition is shown in Table 2, with amounts in wt. %.

TABLE 2
Exemplary composition

| | | Amount |
|---|---|---|
| Phase | Component | (wt. %) |
| 1 | Purified water | 70.68 |
| 1 | Acrylates/C₁₀-C₃₀ alkyl acrylate | 0.20 |
| | crosspolymer | |
| 1 | Disodium EDTA | 0.05 |
| 1 | Chlorphenesin | 0.30 |
| 1 | 1,3-Butylene glycol | 1.50 |
| 2 | dl-alpha-tocopherol acetate | 0.05 |
| 2 | Polypropylene glycol-14 butyl ether | 7.50 |
| 2 | Cetyl alcohol | 3.00 |
| 2 | Stearyl alcohol | 1.50 |
| 2 | Butylated hydroxytoluene | 0.10 |
| 2 | Steareth-21 | 2.00 |
| 2 | Steareth-2 | 1.00 |
| 3 | Triethanolamine | 1.00 |
| 3 | Purified water | 1.00 |
| 4 | Ethoxydiglycol | 4.00 |
| 4 | DeoxyArbutin | 3.00 |
| 5 | Sodium metabisulfite | 0.15 |
| 5 | Magnesium ascorbyl phosphate | 0.10 |
| 5 | Ascorbyl palmitate | 0.10 |
| 5 | Polyaminopropyl biguanide (20%) | 0.50 |
| 5 | Dimethicone | 1.00 |
| 5 | Benzyl alcohol | 0.50 |
| 6 | Triethanolamine | 0.50 |
| 6 | Lactic Acid | 0.01 |
| 7 | Triethanolamine | 0.26 |

The composition in Table 2 was prepared by first filling a kettle with a nitrogen blanket at 15 psi. Water was added and with a high rate of shear, the acrylates/C₁₀-C₃₀ alkyl acrylate crosspolymer (Pemulen TR-1) was added slowly to avoid lump formation. After mixing to generate a clear and uniform solution (20-25 min), the mixture was heated to 75° C. and the remaining phase 1 components were added one at a time and the mixture was stirred until uniform. The components of phase 2 were mixed separately and heated to 75° C., then were added to the kettle and the solution was mixed until uniform. The mixture was cooled to 60° C., the components of phase 3 were pre-mixed and added with stirring. A separate kettle was filled with a nitrogen blanket at 15 psi. The ethoxydiglycol was heated to 45-50° C. and the deoxyArbutin was added slowly under yellow lighting, and slowly mixed until completely dissolved. This mixture was then slowly added to the main kettle, which was mixed until uniform (10-15 min) The batch was then cooled to 45° C. and the phase 5 components were added one at a time with mixing until uniform. The mixture was cooled to 35° C. prior to the addition of benzyl alcohol. The phase 6 components were then added, and the pH was finally adjusted with the addition of phase 7 to reach a final pH of 7.75-7.80.

### Example 3

### Stability Data

Exemplary composition 2 was subjected to stability testing, either in bulk form or in packaged form, at either 25° C. or 40° C. Results are provided in Tables 3 and 4.

TABLE 3
Bulk stability data

| | | Day 8 | Day 31 | Month 2 | Month 3 | Month 6 |
|---|---|---|---|---|---|---|
| Properties | Day 1 | 25° C. | 25° C. | 25° C. | 25° C. | 25° C. |
| Color | off-white | off-white | off-white | off-white | off-white | off-white |
| Odor | characteristic | no change | no change | no change | no change | no change |
| Appearance | opaque, S/V | no change | no change | no change | no change | no change |
| | liquid | | | | | |
| pH | 8.26 | 8.21 | 8.06 | 8.00 | 8.24 | 8.21 |
| Viscosity (RVF | 32000 | 32400 | 32000 | 32800 | 33200 | 29200 |
| #5 @ 10 rpm) | | | | | | |
| DeoxyArbutin | 3.13% | 3.01% | 3.05% | 3.13% | 2.90% | 3.18% |
| Hydroquinone | 0.00% | 0.00% | 0.00% | 0.00% | 0.01% | 0.02% |
| | | Day 8 | Day 31 | Month 2 | Month 3 | Month 6 |
| Properties | Day 1 | 40° C. | 40° C. | 40° C. | 40° C. | 40° C. |
| Color | off-white | off-white | off-white | light beige | beige surface, | dark beige top, |
| | | | | | off-white on | light beige |
| | | | | | rest of the | middle, off- |
| | | | | | bottle | white bottom |
| Odor | characteristic | no change | no change | no change | no change | no change |
| Appearance | opaque, S/V | no change | no change | no change | no change | no change |
| | liquid | | | | | |
| pH | 8.26 | 8.25 | 8.14 | 8.12 | 8.27 | 8.13 |
| Viscosity (RVF | 32000 | 35000 | 42000 | 41000 | 43000 | 37600 |
| #5 @ 10 rpm) | | | (RVF #6 | (RVF #6 | (RVF #6 | |
| | | | @ 10 rpm) | @ 10 rpm) | @ 10 rpm) | |
| DeoxyArbutin | 3.13% | 2.98% | 2.92% | 30.40% | 2.89% | 3.01% |
| Hydroquinone | 0.00% | 0.00% | 0.03% | 0.04% | 0.07% | 0.14% |

TABLE 4
Packaged stability data

| | | Day 8 | Day 31 | Month 2 | Month 3 | Month 6 |
|---|---|---|---|---|---|---|
| Properties | Day 1 | 25° C. | 25° C. | 25° C. | 25° C. | 25° C. |
| Color | off-white | off-white | off-white | off-white | off-white | off-white |
| Odor | characteristic | no change | no change | no change | no change | no change |
| Appearance | opaque, S/V | no change | no change | no change | no change | no change |
| | liquid | | | | | |
| pH | 8.27 | 7.98 | 8.11 | 7.95 | 8.10 | 8.19 |
| Viscosity (RVF | 32400 | 35200 | 30400 | 36800 | 37200 | 37200 |
| #5 @ 10 rpm) | | | | | | |
| External surface | Acceptable | Acceptable | Acceptable | Acceptable | Acceptable | Acceptable |
| Shape | Acceptable | Acceptable | Acceptable | Acceptable | Acceptable | Acceptable |
| Functionality | Acceptable | Acceptable | Acceptable | Acceptable | Acceptable | Acceptable |
| Weight (sample 1) | 82.73 | 82.82 | 82.82 | 82.82 | 82.81 | 82.98 |
| Weight (sample 2) | 82.36 | 82.44 | 82.45 | 82.44 | 82.43 | 82.58 |
| DeoxyArbutin | 2.98% | 2.97% | 2.99% | 3.09% | 2.91% | 3.19% |
| Hydroquinone | 0.00% | 0.00% | 0.02% | 0.01% | 0.02% | 0.02% |
| | | Day 8 | Day 31 | Month 2 | Month 3 | Month 6 |
| Properties | Day 1 | 40° C. | 40° C. | 40° C. | 40° C. | 40° C. |
| Color | off-white | off-white | off-white | off-white | off-white | |
| Odor | characteristic | no change | no change | no change | no change | no change |
| Appearance | opaque, S/V | no change | no change | no change | no change | no change |
| | liquid | | | | | |
| pH | 8.27 | 8.06 | 8.15 | 8.06 | 8.14 | 8.24 |
| Viscosity (RVF | 32400 | 34100 | 41000 | 35200 | 39000 | 37600 |
| #5 @ 10 rpm) | | | | | (RVF #6 | (RVF #6 |
| | | | | | @ 10 rpm) | @ 10 rpm) |
| External surface | Acceptable | Acceptable | Acceptable | Acceptable | Acceptable | Acceptable |
| Shape | Acceptable | Acceptable | Acceptable | Acceptable | Acceptable | Acceptable |
| Functionality | Acceptable | Acceptable | Acceptable | Acceptable | Acceptable | Acceptable |
| Weight (sample 1) | 81.95 | 82.05 | 82.05 | 82.05 | 82.04 | NR |
| Weight (sample 2) | 82.94 | 83.05 | 83.05 | 83.05 | 83.05 | 83.13 |
| DeoxyArbutin | 2.98% | 2.95% | 2.96% | 3.02% | 2.94% | 3.06% |
| Hydroquinone | 0.00% | 0.01% | 0.00% | 0.05% | 0.04% | 0.11% |

Exemplary composition 2 was also subjected to several freeze-thaw cycles, and the product was evaluated after each cycle. Results are provided in Table 5.

TABLE 5
Freeze-thaw data

| | First Cycle | Second Cycle | Third Cycle |
|---|---|---|---|
| Properties | Day 1 | Day 6 | Day 8 |
| Color | off-white | off-white | off-white |
| Odor | no change | no change | no change |
| Appearance | no change | no change | no change |

### Example 4

### Method of Lightening Skin in Hairless Guinea Pig

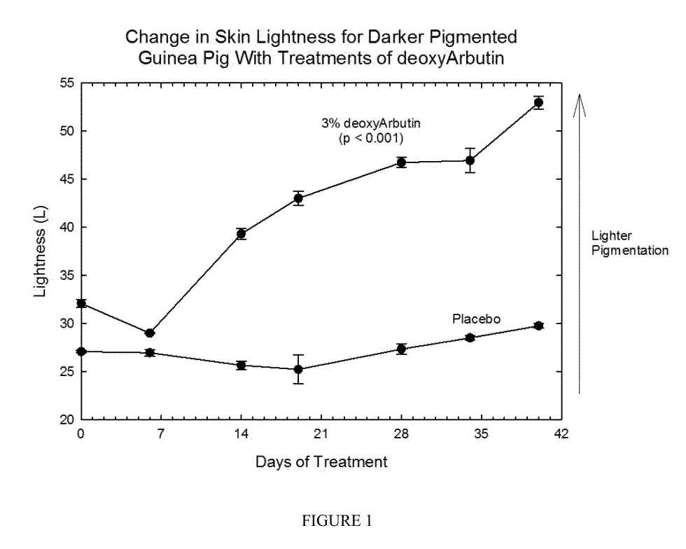

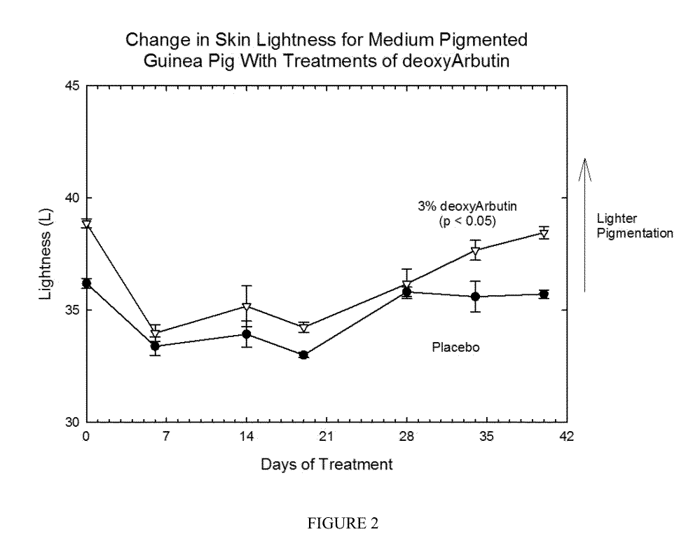

Hairless pigmented guinea pigs with dark or medium skin pigmentation were purchased, housed in cages, and treated according to GLP (good laboratory practices) under Veterinary care. The guinea pigs were treated twice daily with 0.25 g of skin lotions (e.g., placebo or 3% deoxyArbutin formulation 2 of Table 1) on 3.5 cm² treatment areas on the backs of the guinea pigs. All lotions were prepared under GMP (good manufacturing practice) conditions and passed all finished product specifications. At time zero and weekly for 6 weeks, the skin colors (Lab) were read by a Minolta Chromameter on three different areas within the treatment area. The laboratory area had low lightening so to minimize any impact of stray light on the L (Lightness) values. The Lab values were recorded into an Excel spreadsheet. Statistical analyses and graphs were created in Sigma Plot. Results are illustrated in FIGS. 1 and 2 and show lightening of the skin that was treated with the deoxyArbutin composition.

### Example 5

### Method of Lightening Human Skin

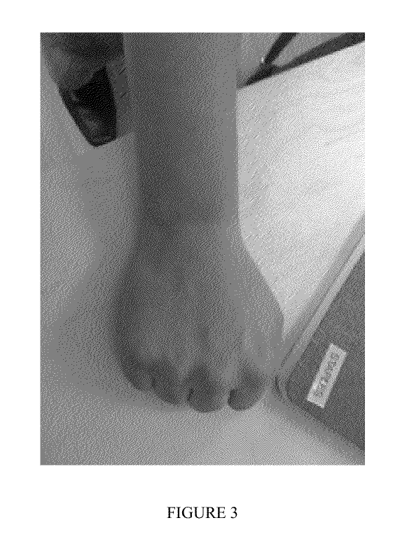

A composition comprising 3% deoxyArbutin, similar to those of Example 1, was applied to the skin of a human subject's hand about 3 times a day for about 3 weeks. A photograph of the subject's hand is illustrated in FIG. 3, and shows lightening of the skin of the hand compared to the skin of the forearm.

### Example 6

### Methods of Lightening Human Skin

A composition of Example 1 will be applied to human skin twice daily, at an amount of about 0.25 g of the composition per application. After about one month, it is expected that a strong skin lightening effect will be observed.

### Example 7

### Methods of Lightening Human Hair

A composition of Example 1 will be applied to human hair twice daily, at an amount of about 0.25 g of the composition per application. After about one month, it is expected that a hair lightening effect will be observed.

### Example 8

### Method of Lightening Skin in Hairless Guinea Pig

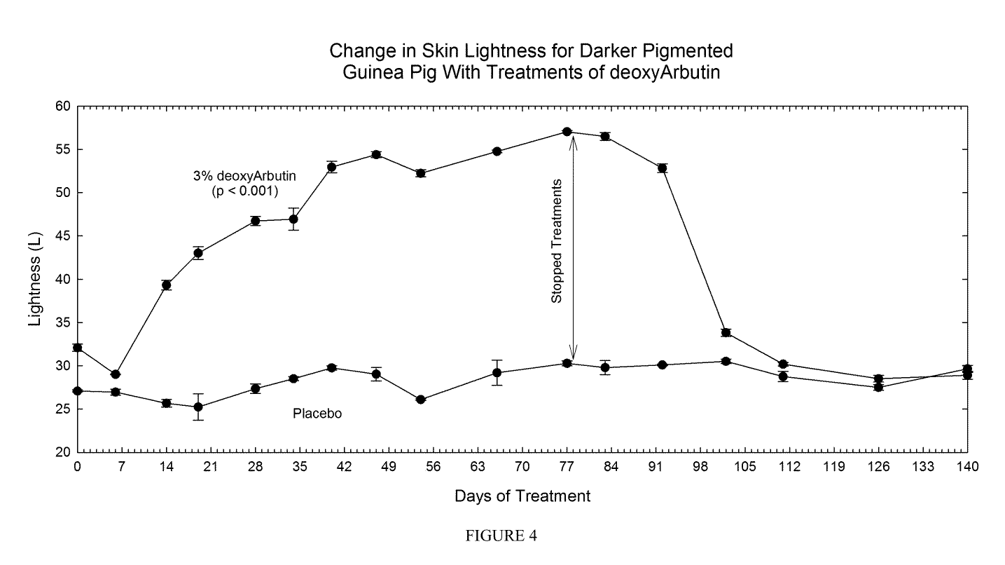

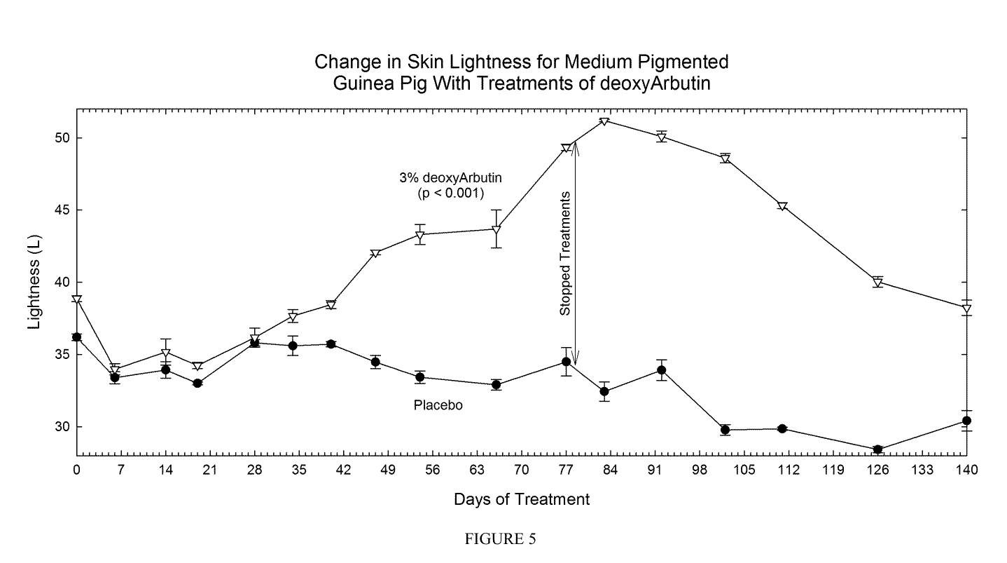

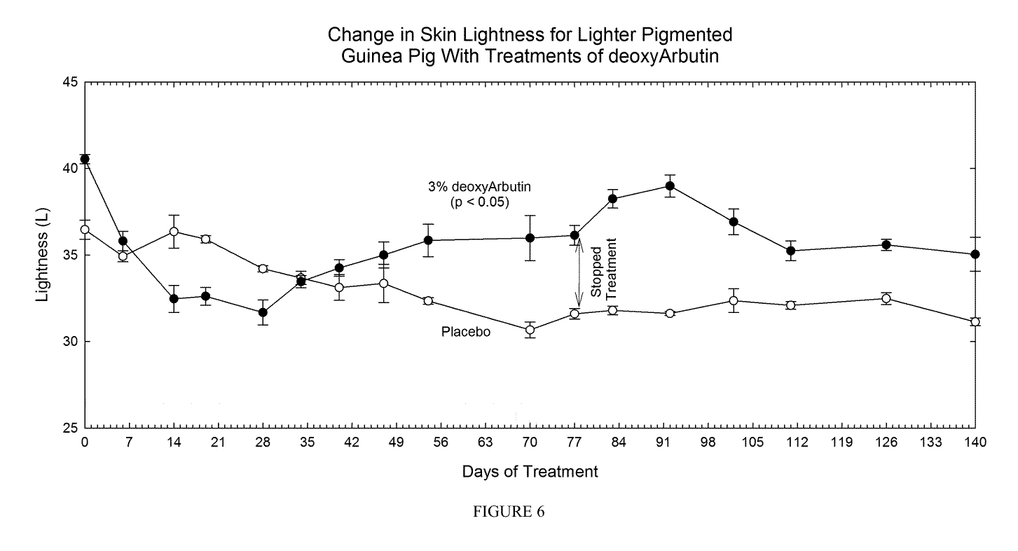

Hairless pigmented guinea pigs with dark or medium skin pigmentation were purchased, housed in cages, and treated according to GLP (good laboratory practices) under Veterinary care. The guinea pigs were treated twice daily with 0.25 g of skin lotions (e.g., placebo or 3% deoxyArbutin formulation 2 of Table 1) on 3.5 cm² treatment areas on the backs of the guinea pigs. All lotions were prepared under GMP (good manufacturing practice) conditions and passed all finished product specifications. At time zero and weekly for 11 weeks, the skin colors (Lab) were read by a Minolta Chromameter on three different areas within the treatment area. The laboratory area had low lightening so to minimize any impact of stray light on the L (Lightness) values. Following the 11 weeks, the twice daily treatments were stopped, while the skin colors were recorded for another 9 weeks. The Lab values were recorded into an Excel spreadsheet. Statistical analyses and graphs were created in Sigma Plot. Results are illustrated in FIGS. 4-6 and show lightening of the skin that was treated with the deoxyArbutin composition, followed by a return toward the original pigmentation once treatments were stopped.

Although the disclosure above has been described in terms of various aspects and specific embodiments, it is not so limited. A variety of suitable alterations and modifications for operation under specific conditions will be apparent to those skilled in the art. It is therefore intended that the following claims be interpreted as covering all such alterations and modifications as fall within the spirit and scope of the invention.

All patents, publications and references cited herein are hereby fully incorporated by reference. In case of conflict between the present disclosure and incorporated patents, publications and references, the present disclosure should control.

1. A composition comprising:
a) a compound of formula I:
or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur; and
b) at least one of an antioxidant, a glycol and a solvent;
wherein the composition comprises less than 100 ppm O₂, and wherein the composition exhibits a change in L* value of less than about 10.0 when stored at ambient temperature and pressure for a period of time, wherein the period of time is at least about 2 days.

2. The composition of claim 1, wherein Z is O.

3. The composition of claim 1, wherein A and B are taken together with the atoms to which they are attached to form an optionally substituted ring having from 4-9 member atoms.

4. The composition of claim 1, wherein A and B are taken to taken together with the atoms to which they are attached to form a ring having 6 member atoms.

5. The composition of claim 1, wherein the compound has the formula I-a:
or a pharmaceutically acceptable salt thereof, wherein:
n is 1, 2, 3, 4 or 5;
each R is independently selected from hydrogen and C1-6 aliphatic; and
each R′ is independently selected from hydrogen, C1-6 aliphatic and —OR^{a}, wherein each R^{a} is independently selected from hydrogen and C1-6 aliphatic.

6. The composition of claim 1, wherein the compound has the formula I-b:

7. The composition of claim 1, wherein the compound has the formula I-c:

8. The composition of claim 1, wherein the compound has the formula I-d:
wherein R^{a} and R^{b} are each independently selected from the group consisting of hydrogen, optionally substituted C1-6 aliphatic, and —OR², wherein R² is selected from the group consisting of hydrogen and optionally substituted C1-6 aliphatic.

9. The composition of claim 1, wherein the compound has the formula I-e:

10. The composition of claim 1, wherein the compound has the formula I-f:
wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from the group consisting of: C1-6 aliphatic; a 3-8 membered saturated carbocyclic ring; and phenyl.

11. The composition of claim 1, wherein the compound of formula I is selected from:

12. The composition of claim 1, wherein the compound of formula I is 4-(tetrahydro-2H-pyran-2-yloxy)phenol.

13. The composition of claim 1, wherein the compound of formula I is present in the composition at a concentration of about 0.5 wt. % to about 10 wt. %.

14. The composition of claim 1, wherein the composition comprises an antioxidant and the antioxidant comprises at least one of ascorbic acid, tocopherol, butylated hydroxybenzoic acid, butylated hydroxytoluene, butylated hydroxyanisole, uric acid, gallic acid, sorbic acid, glutathione, and esters and salts of any thereof.

15. The composition of claim 1, wherein the composition comprises an antioxidant and the antioxidant comprises at least one of ascorbic acid or a salt thereof, ascorbyl phosphate or a salt thereof, ascorbyl palmitate or a salt thereof, tocopherol or a salt thereof, tocopheryl acetate and sodium metabisulfite.

16. The composition of claim 1, comprising at least two antioxidants.

17. The composition of claim 1, comprising at least three antioxidants.

18. The composition of claim 1, comprising at least four antioxidants.

19. The composition of claim 1, comprising at least five antioxidants.

20. The composition of claim 1, wherein the composition comprises an antioxidant and the antioxidant is present in an amount of about 0.01 wt. % to about 3.0 wt. %.

21. The composition of claim 1, wherein the composition comprises an antioxidant and the antioxidant is present in an amount of about 0.05 wt. % to about 0.6 wt. %.

22. The composition of claim 1, wherein the composition comprises a glycol and the glycol is present in an amount of from about 0.1 wt. % to about 10 wt. %.

23. The composition of claim 1, wherein the composition comprises a glycol and the glycol comprises ethoxydiglycol.

24. The composition of claim 1, further comprising at least one additional component selected from emulsifiers, chelating agents, preservatives, solvents, conditioning agents, pH adjusters, anti-inflammatory agents, sunscreens, retinoids, anti-aging agents, exfoliants and anti-acne agents.

25. The composition of claim 1, wherein the composition comprises less than 15 ppm O₂.

26. The composition of claim 1, wherein the period of time is at least about 7 days.

27. The composition of claim 1, wherein the period of time is at least about 10 days.

28. The composition of claim 1, wherein the composition is substantially free of dyes and pigments.

29. The composition of claim 1, wherein the composition exhibits a change in L* value of less than about 5.0 over the period of time.

30. The composition of claim 1, wherein the composition is substantially free of hydroquinone.

31. A composition comprising:
a) a compound of formula I:
or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur;
b) an antioxidant; and
c) ethoxydiglycol;
wherein the composition comprises less than 100 ppm O₂.

32.-63. (canceled)

64. A method of preparing a composition, the method comprising:
mixing a compound of formula I:
or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur
with at least one of an antioxidant, a glycol and a solvent under an inert atmosphere; and
maintaining the pH at a range of about 6.0 to about 10.0.

65.-104. (canceled)

105. A composition comprising:
a) about 0.5 wt. % to about 10 wt. % of a compound of formula I:
or a pharmaceutically acceptable salt thereof, wherein:
Z is selected from NR₁, S, O, SO and SO₂,
R₁ is selected from hydrogen and C1-6 aliphatic;
X₁ and X₂ are independently selected from hydrogen, halogen, cyano, nitro, optionally substituted C1-6 aliphatic, —OR², —SR², —N(R²)₂, —COOR², and —CON(R²)₂;
each R² is independently hydrogen or an optionally substituted C1-6 aliphatic; and
A and B are independently an optionally substituted group selected from C1-10 aliphatic, a 3-8 membered saturated or partially unsaturated carbocyclic ring, a 3-8 membered saturated or partially unsaturated heterocyclic ring having 1-2 heteroatoms independently selected from nitrogen, oxygen, and sulfur, phenyl, an 8-10 membered bicyclic aryl ring, a 5-6 membered heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, or sulfur;
or A and B may be taken together with the atoms to which they are attached to form an optionally substituted saturated or partially unsaturated monocyclic or bicyclic ring having from 4-12 member atoms and 0-2 heteroatoms independently selected from nitrogen, oxygen, and sulfur;
b) about 0.01 wt. % to about 3.0 wt. % of at least one antioxidant;
c) about 0.1 wt. % to about 10 wt. % of at least one glycol;
d) about 0.01 wt. % to about 10 wt. % of at least one emulsifier; and
e) water;
wherein the composition comprises less than 100 ppm O₂.

106.-145. (canceled)
